(19) United States
(12) Patent Application Publication
(10) Pub. No.: US 2003/0082806 A1
(43) Pub. Date: May 1, 2003

(54) Maturation of antigen-presenting cells using activated T cells

(75) Inventors: Ronald Jay Berenson, Mercer Island, WA (US); Mark Bonyhadi, Issaquah, WA (US); Stewart Craig, Issaquah, WA (US); Dale Kalamasz, Redmond, WA (US); Tatsue Monji, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: XCYTE Therapies, Inc., Seattle, WA

(21) Appl. No.: 10/136,024

(22) Filed: Apr. 29, 2002

Related U.S. Application Data
(60) Provisional application No. 60/287,168, filed on Apr. 27, 2001.
(60) Provisional application No. 60/295,331, filed on Jun. 1, 2001.

(51) Int. Cl. A61K 48/00; C12N 5/08

(52) U.S. Cl. 435/372; 424/93.21

(57) ABSTRACT

The present invention relates to methods for maturing antigen-presenting cells, and more particularly, to methods for maturing dendritic cells. Methods for generating mature and/or maturing antigen-presenting cells in vitro and in vivo are disclosed. The present invention also relates to compositions of cells, including mature antigen-presenting cells and/or activated T cells and their use in generating immune responses in vivo, and inhibiting the development of or preventing infectious diseases and cancers.

### TECHNICAL FIELD

The present invention relates generally to methods for maturing antigen-presenting cells (APC), and more particularly, to methods to mature APC such as dendritic cells (DC). The present invention also relates to compositions of cells, including mature APC and/or activated T cells.

### BACKGROUND OF THE INVENTION

Presentation of antigen to T cells is a central step in the process of immune activation. Numerous cell types have the capacity to present antigen, including DC, macrophages, and activated B cells. Numerous organ-specific cell populations, for example Kuppfer cells in the liver, and Langerhans cells in the skin are subpopulations of DC. Not all APC are equally effective, and it is generally accepted that DC are the most potent APC. (William E. Paul (ed.), Fundamental Immunology, 4^{th} ed., Lippincoft-Raven Publishers, New York, 1999).

DC are APC that function to initiate several immune responses such as the sensitization of MHC-restricted T cells, the rejection of organ transplants, and the formation of T cell-dependent antibodies. DC are found in many non-lymphoid tissues but can migrate via the afferent lymph or the blood stream to the T cell-dependent areas of lymphoid organs. They are found in the skin, where they are named Langerhans cells, and are also present in the mucosa. They represent the sentinels of the immune system within the peripheral tissues where they can acquire antigens. As these cells often express CD4 and can be infected in vitro by HIV, they are likely to present a port of entry of virus in vivo: e.g., Knight et al., pp. 145 in Racz et al. (eds.), Accessory Cells in HIV and Other Retroviral Infections (Karger, Basel, 1991); Ramsauer et al., pp. 155 in Racz et al. (eds.) (cited above). The isolation of human DC from peripheral blood has only recently been achieved and only small numbers of cells can be generated, e.g., Freudenthal et al., Proc. Natl. Acad. Sci. 87:7698, 1990. The in vitro generation of larger numbers of human DC, and DC that function more effectively, would present an important advantage for generating in vivo primary and secondary immune responses and for priming in vitro human naive CD4⁺ and CD8⁺ T cells.

### SUMMARY OF THE INVENTION

The present invention generally provides methods for maturing cells, and more particularly, provides a novel method to mature DC. One aspect of the present invention provides a method for maturing DC, comprising providing a population of cells wherein at least a portion thereof comprises immature DC; and exposing the population of cells to activated T cells or supernatant therefrom, thereby inducing maturation.

In one embodiment of the method, the immature DC are generated from a source of precursor cells that may comprise leukapheresis product, peripheral blood, lymph node, skin, gut associated lymphoid tissue (GALT), tonsil, thymus, tissue biopsy, tumor, spleen, bone marrow, cord blood, CD34⁺ cells, monocytes, or adherent cells, or any combination thereof.

In another embodiment of the method, the immature DC are generated from any one or a combination of these sources of precursor cells, by exposing said precursor cells to one or more cytokines. In a further embodiment, the cytokines may comprise granulocyte-macrophage colony-stimulating factor (GM-CSF), interleukin 4 (IL-4), and IL-13, or any combination thereof.

In another embodiment of the method, the immature DC are generated by exposing precursor cells to one or more cytokines as described above, and to activated T cells and/or supernatant therefrom.

In one embodiment of the method, the immature DC are loaded with antigen through gene modification or by exposing the immature DC to a source of antigen that may comprise protein, glycoprotein, peptides, antibody/antigen complexes, tumor lysate, non-soluble cell debris, apoptotic bodies, necrotic cells, whole tumor cells from a tumor or a cell line that have been treated such that they are unable to continue dividing, allogeneic cells that have been treated such that they are unable to continue dividing, irradiated tumor cells, irradiated allogeneic cells, natural or synthetic complex carbohydrates, lipoproteins, lipopolysaccharides (LPS), transformed cells or cell line, transfected cells or cell line, or transduced cells or cell line, or any combination thereof.

In another embodiment, the immature DC are genetically modified.

In a further embodiment of the method, the immature DC are generated by administering to a mammal a composition comprising a compound that increases the number of DC in the blood and a pharmaceutically acceptable excipient. In one preferred embodiment, the compound may comprise Flt3-ligand (Flt3-L) or CD40 ligand (CD40L).

The present invention also provides for populations of mature DC generated according to any of the methods described herein.

In one embodiment of the method, the activated T cells comprise a T cell line.

In another embodiment the activated T cells are generated by cell surface moiety ligation comprising providing a population of cells wherein at least a portion thereof comprises T cells, and exposing the population of cells to an agent or agents that induce the desired activation. In one embodiment, the agent may comprise anti-CD3 antibodies, anti-CD28 antibodies, peptide-MHC tetramers, or superantigens, or a combination thereof.

In one embodiment of the method, the activated T cells are generated by exposing to a mitogen a population of cells wherein at least a portion thereof comprises T cells. In one preferred embodiment, the mitogen may comprise phytohemagglutinin (PHA), phorbol myristate acetate (PMA) and ionomycin, lipopolysaccharide (LPS), or a combination thereof.

In one embodiment of the methods, the activated T cells are generated by simultaneous T cell concentration and cell surface moiety ligation, comprising: providing a population of cells wherein at least a portion thereof comprises T cells; exposing the population of cells to a surface, wherein the surface has attached thereto one or more agents that ligate a cell surface moiety of at least a portion of the T cells and stimulate at least the portion of T cells; with the option, but not a requisite option, of applying a force that predominantly drives T cell concentration and T cell surface moiety ligation, thereby inducing T cell stimulation.

The present invention also provides a composition comprising the DC generated according to the above methods and a pharmaceutically acceptable excipient. In one embodiment, the composition may comprise DC that have been genetically modified.

In one embodiment, a method is provided for stimulating an immune response in a mammal comprising, administering to the mammal a composition of DC of the present invention. In a preferred embodiment, the immune response comprises the activation of T cells in the mammal.

In another embodiment, a method is provided for ameliorating an immune response dysfunction in a mammal comprising administering to the mammal a composition of mature DC of the present invention. In yet another embodiment, a method is provided for reducing the presence of cancer cells in a mammal comprising, exposing the cancer cells to the composition of DC. In one embodiment, the cancer cells may comprise cells from melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, pancreatic cancer, esophageal cancer, brain cancer, lung cancer, ovarian cancer, cervical cancer, multiple myeloma, hepatocellular carcinoma, nasopharyngeal carcinoma, ALL, AML, CML, or CLL, or a combination thereof.

A further embodiment provides a method for reducing the presence of an infectious organism in a mammal comprising, administering a composition of the present invention to the mammal. In one preferred embodiment, the infectious organism may comprise a virus, such as a single stranded RNA virus or a single stranded DNA virus, human immunodeficiency virus (HIV), hepatitis A, B, or C virus, herpes simplex virus (HSV), human papilloma virus (HPV), cytomegalovirus (CMV), Epstein-Barr virus (EBV), a parasite, a bacterium, M. tuberculosis, Pneumocystis carinii, Candida, or Aspergillus or a combination thereof.

In another embodiment, a method is provided for inhibiting the development of a cancer in a mammal, comprising administering to the mammal a composition of DC of the present invention. In a further embodiment, the cancer may comprise melanoma, non-Hodgkin's lymphoma, Hodgkin's disease, leukemia, plasmocytoma, sarcoma, glioma, thymoma, breast cancer, prostate cancer, colorectal cancer, kidney cancer, renal cell carcinoma, pancreatic cancer, esophageal cancer, brain cancer, lung cancer, ovarian cancer, cervical cancer, multiple myeloma, hepatocellular carcinoma, nasopharyngeal carcinoma, ALL, AML, CML, or CLL or a combination thereof.

In another embodiment, a method is provided for inhibiting the development of an infectious disease in a mammal, comprising administering to the mammal a composition of DC of the present invention. In a further embodiment, the infectious disease may comprise a disease caused by a virus such as a single stranded RNA virus, a single stranded DNA virus, a double-stranded DNA virus, HIV, Hepatitis A, B, or C, virus, HSV, CMV, EBV, a parasite, a bacterium, M. tuberculosis, Pneumocystis carinii, Candida, or Aspergillus or a combination thereof.

One aspect of the present invention provides a composition comprising DC and activated T cells Wherein the DC have been matured by exposure to activated T cells and/or supernatant therefrom ex vivo. In one embodiment, the composition also comprises a pharmaceutically acceptable excipient.

In one embodiment, a method is provided for stimulating an immune response in a mammal, comprising administering to the mammal a composition of DC and activated T cells as generated by the present invention.

In another embodiment, a method is provided for reducing the presence of an infectious organism, as listed above, in a mammal comprising administering to the mammal a composition of DC and activated T cells.

In a further embodiment, a method is provided for inhibiting the development of any of the above-mentioned cancers in a mammal, comprising administering to the mammal a composition of DC and activated T cells of the present invention.

Another embodiment provides a method for inhibiting the development of any of the above-listed infectious diseases in a mammal, comprising administering to the mammal a composition of DC and activated T cells of the present invention.

Another aspect of the present invention provides a method for reducing the presence of cancer cells in a mammal, comprising administering to the mammal a composition comprising, DC matured by activated T cells and/or supernatant therefrom ex vivo, activated T cells, and a pharmaceutically acceptable excipient, wherein the cancer cells may comprise cells from any of the cancers listed above.

One aspect of the present invention provides a method for inducing DC maturation in vivo, comprising: administering a population of cells to a mammal wherein at least a portion of the population comprises immature DC generated ex vivo; administering particles to a mammal, wherein at least one portion of the particles has attached thereto, ligands specific for a T cell moiety that induces T cell activation, wherein a second portion of the particles has attached thereto, ligands specific for a DC surface moiety; inducing co-localization of the T cells and the DC, to achieve activation of the T cells and desired maturation of the DC. In one embodiment of the method, the particles are paramagnetic. In yet another embodiment, the co-localization is achieved by applying a magnetic field to a discrete region of the mammal. In yet a another embodiment, the particles further comprise ligands specific for a discrete tissue of the mammal. In a further embodiment, the discrete tissue may comprises a tumor, lymph node tissue, mucosal lymphoid tissue gut associated lymphoid tissue (GALT), or skin, or any combination thereof.

One aspect of the present invention provides a method for generating mature DC in vivo comprising, administering to a mammal a composition comprising activated T cells.

Another aspect of the invention provides a method for generating DC in vivo, comprising, administering to a mammal a composition comprising a compound that increases the number of DC in the blood and a pharmaceutically acceptable excipient. In one embodiment, the compound may comprise Flt3-L, soluble CD40L, GM-CSF and IL-4 or IL-13, or any combination thereof. In another embodiment, the method further comprises the administration of activated T cells.

Another aspect of the invention provides a method for generating mature DC that comprises: generating immature DC in vitro from a source of precursor cells by a method that may comprise, i. exposing the precursor cells to GM-CSF and IL-4; ii. exposing the precursor cells to GM-CSF and IL-13; iii. exposing the precursor cells to activated T cells; iv. exposing the precursor cells to activated T cell supernatant; v. exposing the precursor cells to GM-CSF and IL-4 and activated T cells; vi. exposing the precursor cells to GM-CSF, IL-4, and activated T cell supernatant; vii. exposing the precursor cells to GM-CSF and IL-13 and activated T cells; or viii. Exposing the precursor cells to GM-CSG, IL-13, and activated T cell supernatant; administering to a mammal the immature DC, and; administering to the mammal activated T cells, thereby inducing in vivo maturation of the immature DC. In one embodiment of the method the source of precursor cells may comprise leukapheresis product, peripheral blood, lymph node, skin, GALT, tonsil, thymus, tissue biopsy, tumor, spleen, bone marrow, cord blood, CD34⁺ selected cells, monocytes, or adherent cells, or a combination thereof.

Another aspect of the invention provides a method for generating mature DC that comprises: obtaining a population of cells from a mammal wherein at least a portion thereof comprises precursor DC; exposing said portion of cells in vitro to GM-CSF and IL-4 or IL-13 to generate immature DC; exposing said immature DC in vitro to a population of activated T cells for a sufficient period of time to achieve desired maturation. In one embodiment, the precursor cells may be isolated from peripheral blood. In a further embodiment the precursor cells may be isolated from leukapheresis product. In a further embodiment, the activated T cells may be generated by a method that comprises exposing the population of T cells to an anti-CD3 antibody and a ligand which binds an accessory molecule on the surface of the T cells, under conditions appropriate for activation of the T cells. In a further embodiment, the activated T cells may be generated by a method that comprises, exposing the population of T cells to an anti-CD3 antibody which is immobilized on a solid phase surface; and; stimulating an accessory molecule on the surface of the T cells with an anti-CD28 antibody, wherein said anti-CD28 antibody is immobilized on the same solid phase surface as the anti-CD3 antibody, thereby inducing activation and proliferation of the T cells. In one embodiment, the activated T cells generated by this method comprise T cells that have proliferated. In another embodiment, the activated T cells generated by this method comprise T cells that secrete cytokines.

The present invention is not only applicable to maturing DC but can be used in all its aspects and embodiments for maturing other APC.

The present invention provides methods and embodiments thereof, that comprise activated T cells or supernatant therefrom to mature DC and other APC. In any of these methods and embodiments thereof, activated T cells or supernatant therefrom may be used.

### BRIEF DESCRIPTION OF THE DRAWINGS

FIG. 1 is a plot depicting a time course analysis of the concentration of IL-2 in the culture supernatant during the XCELLERATE™ process.

FIG. 2 is a plot depicting a time course analysis of the concentration of IL-4 in the culture supernatant during the XCELLERATE™ process.

FIG. 3 is a plot depicting a time course analysis of the concentration of tumor necrosis factor-alpha (TNF-α) in the culture supernatant during the XCELLERATE™ process.

FIG. 4 is a plot depicting a time course analysis of the concentration of interferon-gamma (IFN-γ) in the culture supernatant during the XCELLERATE™ process.

FIG. 5 is a plot depicting a time course analysis of the levels of CDw137 (4-1 BB) expression in XCELLERATE™ activated T cells.

FIG. 6 is a plot depicting a time course analysis of the levels of CD154 (CD40L) expression in XCELLERATE™ activated T cells.

FIG. 7 is a plot depicting a time course analysis of the levels of CD25 expression in XCELLERATE™ activated T cells.

FIG. 8 contains 4 panels depicting the expression levels of DR, CD86, Lineage (CD3, CD14, CD16, CD19, CD20, and CD56), and CD14 in precursor cells cultured in the presence of XCELLERATE™ cell supernatant as compared to the levels of these markers in precursor cells cultured with media alone.

FIG. 9 contains 4 histogram plots measuring expression levels of CD80, CD83, CD86, and HLA-DR on DC matured in the presence day 2 or day 3 XCELLERATE™ activated T cells.

FIG. 10 is a photograph of multinucleated cells resulting from co-culture of day 1 to day 2 monocytes with day 2 or day 3 XCELLERATE™ activated T cells for 3-4 days.

FIG. 11 is a graph depicting the concentration of various cytokines in the T cell culture supernatant on day 3 of the XCELLERATE™ process.

### DETAILED DESCRIPTION OF THE INVENTION

Prior to setting forth the invention, it may be helpful to an understanding thereof to set forth definitions of certain terms that will be used hereinafter.

The term “biocompatible,” as used herein, refers to the property of being predominantly non-toxic to living cells.

The term “stimulation,” as used herein, refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event, such as binding the TCR/CD3 complex. Further, the stimulation event may activate a cell and up or downregulate expression or secretion of a molecule, such as downregulation of Tumor Growth Factor beta (TGF-β). Thus, ligation of cell surface moieties, even in the absence of a direct signal transduction event, may result in the reorganization of cytoskeletal structures, or in the coalescing of cell surface moieties, each of which could serve to enhance, modify, or alter subsequent cell responses.

The term “activation,” as used herein, refers to the state of a cell following sufficient cell surface moiety ligation to induce a measurable morphological, phenotypic, and/or functional change. Within the context of T cells, such activation may be the state of a T cell that has been sufficiently stimulated to induce cellular proliferation. Activation of a T cell may also induce cytokine production and/or secretion, and performance of regulatory or cytolytic effector functions. Within the context of other cells, this term infers either up- or down-regulation of a particular physico-chemical process.

The term “target cell,” as used herein, refers to any cell that is intended to be stimulated by cell surface moiety ligation.

An “antibody,” as used herein, includes both polyclonal and monoclonal antibodies (mAb); primatized (e.g., humanized); murine; mouse-human; mouse-primate; and chimeric; and may be an intact molecule, a fragment thereof (such as scFv, Fv, Fd, Fab, Fab′ and F(ab)′₂ fragments), or multimers or aggregates of intact molecules and/or fragments; and may occur in nature or be produced, e.g., by immunization, synthesis or genetic engineering; an “antibody fragment,” as used herein, refers to fragments, derived from or related to an antibody, which bind antigen and which in some embodiments may be derivatized to exhibit structural features that facilitate clearance and uptake, e.g., by the incorporation of galactose residues. This includes, e.g., F(ab), F(ab)′₂, scFv, light chain variable region (V_{L}), heavy chain variable region (V_{H}), and combinations thereof.

The term “protein,” as used herein, includes proteins, glycoproteins and other cell-derived modified proteins, polypeptides and peptides; and may be an intact molecule, a fragment thereof, or multimers or aggregates of intact molecules and/or fragments; and may occur in nature or be produced, e.g., by synthesis (including chemical and/or enzymatic) or genetic engineering.

The term “agent,” “ligand,” or “agent that binds a cell surface moiety,” as used herein, refers to a molecule that binds to a defined population of cells. The agent may bind any cell surface moiety, such as a receptor, an antigenic determinant, or other binding site present on the target cell population. The agent may be a protein, peptide, antibody and antibody fragments thereof, fusion proteins, synthetic molecule, an organic molecule (e.g., a small molecule), or the like. Within the specification and in the context of T cell stimulation, antibodies are used as a prototypical example of such an agent.

The term “cell surface moiety” as used herein may refer to a cell surface receptor, an antigenic determinant, or any other binding site present on a target cell population.

The terms “agent that binds a cell surface moiety” and “cell surface moiety,” as used herein, should be viewed as a complementary/anti-complementary set of molecules that demonstrate specific binding, generally of relatively high affinity.

A “co-stimulatory signal,” as used herein, refers to a signal, which in combination with a primary signal, such as TCR/CD3 ligation, leads to T cell proliferation and/or activation.

“Separation,” as used herein, includes any means of substantially purifying one component from another (e.g., by filtration, affinity, buoyant density, or magnetic attraction).

A “surface,” as used herein, refers to any surface capable of having an agent attached thereto and includes, without limitation, metals, glass, plastics, co-polymers, colloids, lipids, cell surfaces, and the like. Essentially any surface that is capable of retaining an agent bound or attached thereto.

“Precursor” or “progenitor” cells, as used herein, refer to cells with the capacity to differentiate into multiple, distinct subsets of mature cells, depending on in vivo or in vitro conditions. Examples of precursor or progenitor cells include, but are not limited to, CD34⁺ cells, monocytes, and pre-B cells.

“Immature,” as used herein, refers to a cell differentiation state between the progenitor or precursor and mature states.

“Maturing,” as used herein, refers to the process by which a precursor or progenitor cell differentiates to a mature state. Numerous stages exist along the maturation pathway from progenitor to mature cell, including an immature stage. According to the present invention, maturation may occur in vivo or in vitro or both. For example, precursor cells may be isolated from a tissue sample and matured in vitro. Alternatively, already immature cells may be isolated from a sample and further matured in vitro. Precursor cells may be isolated from a sample, partially matured in vitro, reinfused into an individual and continued maturation carried out in vivo.

“Professional APC” (pAPC) or “antigen-presenting cell” (APC), as used herein, refers to those cells that normally initiate the responses of naive and/or memory T cells to antigen. Professional APCs include, but are not limited to, DC, macrophages, and B cells. pAPC may express high levels of MHC class II, ICAM-1 and B7-2.

“Mature (p)APC” as used herein, refers to the state of an APC following in vitro or in vivo differentiation in the presence of appropriate stimuli such that the mature APC has the capacity to initiate or engage in an immune response. Mature APC, according to the present invention, are characterized by the capacity to prime naive T cells. Further, mature APC may express CD40, CD54, CD80, CD83, CD86, CCR7, ICAM-1, CD1a, and high levels of MHC class II, as measured by mAb staining and flow cytometric analysis.

“Immature APC” as used herein, refers to an intermediate differentiation state of an APC wherein the APC has the capacity to endocytose or phagocytose antigen, foreign bodies, necrotic and/or apoptosing tissue and/or cells. Immature APC may be CD14⁻ or CD14⁺ depending on the origin of the precursor cells. Immature APC may also express CD1 a, CD40, CD86, CD54, and intermediate levels of MHC class II (levels of marker expression on sample cells can be compared by flow cytometric analysis to levels of expression on MHC class II-negative cells and cells known to express high levels of MHC class II). Immature APC typically do not express CCR7.

“Immune response” as used herein, refers to activation of cells of the immune system, including but not limited to, T cells, such that a particular effector function(s) of a particular cell is induced. Effector functions may include, but are not limited to, proliferation, secretion of cytokines, secretion of antibodies, expression of regulatory and/or adhesion molecules, and the ability to induce cytolysis.

“Stimulating an immune response” as used herein, refers to any stimulation such that activation and induction of effector functions of cells of the immune system are achieved.

“Immune response dysfunction” as used herein, refers to the inappropriate activation and/or proliferation, or lack thereof, of cells of the immune system, and/or the inappropriate secretion, or lack thereof, of cytokines, and/or the inappropriate or inadequate induction of other effector functions of cells of the immune system, such as expression of regulatory, adhesion, and/or homing receptors, and the induction of cytolysis.

The terms “preventing” or “inhibiting” the development of a cancer or cancer cells” as used herein, means the occurrence of the cancer is prevented or the onset of the cancer is delayed.

The term “treating” or “reducing the presence of a cancer or cancer cells” as used herein, means that the cancer growth is inhibited, which is reflected by, e.g., tumor volume or numbers of malignant cells. Tumor volume may be determined by various known procedures, e.g., obtaining two dimensional measurements with a dial caliper.

“Preventing or inhibiting the development of an infectious disease” as used herein, means the occurrence of the infectious disease is prevented or the onset of the infectious disease is delayed, or the spread of an existing infection is reversed.

“Ameliorate” as used herein, is defined as: to make better; improve (The American Heritage College Dictionary, 3rd ed. Houghton Mifflin Company, 2000).

“Particles” as used herein, may include a colloidal particle, a microsphere, nanoparticle, a bead, or the like. In the various embodiments, commercially available surfaces, such as beads or other particles, are useful (e.g., Miltenyi Particles, Miltenyi Biotec, Germany; Sepharose beads, Pharmacia Fine Chemicals, Sweden; DYNABEADS™, Dynal Inc., New York; PURABEADS™, Prometic Biosciences, magnetic beads from Immunicon, Huntingdon Valley, Pa., microspheres from Bangs Laboratories, Inc., Fishers, Ind.).

“Paramagnetic particles” as used herein, refer to particles, as defined above, that localize in response to a magnetic field.

“Antigen” as used herein, refers to any molecule 1) capable of being specifically recognized, either in its entirety or fragments thereof, and bound by the “idiotypic” portion (antigen-binding region) of a mAb or its derviative; 2) containing peptide sequences which can be bound by MHC molecules and then, in the context of MHC presentation, can specifically engage its cognate T cell antigen receptor.

To “load” an APC with antigen, as used herein, refers to exposing an APC to antigen or antigenic peptide for a period of time sufficient for the APC to take up, process, and present the antigen, bound by MHC molecules, to T cells. In some cases, the antigen can be bound by MHC molecules and presented to T cells without being taken up and processed by the APC.

The term “animal” or “mammal” as used herein, encompasses all mammals, including humans. Preferably, the animal of the present invention is a human subject.

The term “exposing” as used herein, refers to bringing into the state or condition of immediate proximity or direct contact.

The term “lysate” as used herein, refers to the supernatant and non-soluble cell debris resulting from lysis of cells. A skilled artisan will recognize that any number of lysis buffers known in the art may be used (see, for example, Current Protocols in Immunology, John Wiley & Sons, New York, N.Y.). Cell lysis may also be carried out by freeze-thaw procedures.

The term “apoptotic body” as used herein, is defined as the smaller, intact, membrane-bound fragments that result from apoptotic cells.

The term “proliferation” as used herein, means to grow or multiply by producing new cells.

The term “infectious disease” as used herein, refers to any disease that is caused by an infectious organism. Infectious organisms may comprise viruses, (e.g., single stranded RNA viruses, single stranded DNA viruses, HIV, hepatitis A, B, and C virus, HSV, CMV EBV, HPV), parasites (e.g., protozoan and metazoan pathogens such as Plasmodia species, Leishmania species, Schistosoma species, Trypanosoma species), bacteria (e.g., Mycobacteria, in particular, M. tuberculosis, Salmonella, Streptococci, E. coli, Staphylococci), fungi (e.g., Candida species, Aspergillus species), Pneumocystis carinii, and prions (known prions infect animals to cause scrapie, a transmissible, degenerative disease of the nervous system of sheep and goats, as well as bovine spongiform encephalopathy (BSE), or “mad cow disease,” and feline spongiform encephalopathy of cats. Four prion diseases known to affect humans are (1) kuru, (2) Creutzfeldt-Jakob Disease (CJD), (3) Gerstmann-Straussler-Scheinker Disease (GSS), and (4) fatal familial insomnia (FFI)). As used herein “prion” includes all forms of prions causing all or any of these diseases or others in any animals used—and in particular in humans and domesticated farm animals.

The term “gut associated lymphoid tissue” or “GALT,” as used herein, refers to the lymphoid tissues closely associated with the gastrointestinal tract, including the palatine tonsils, Peyer's patches, and intraepithelial lymphocytes.

The term “mucosal lymphoid tissue” as used herein, refers to all lymphoid cells in epithelia and in the lamina propria lying below the body's mucosal surfaces.

Sources of Antigen-Presenting Cells (APC)

The starting material for the method of producing immature APC (APC) and mature APC is typically a tissue source comprising APC precursors that are capable of proliferating and maturing in vitro into professional APC (pAPC) when treated according to the method of the invention. In one aspect, APC precursor cells are capable of proliferating and maturing in vitro into DC (DC). While many tissue sources may be used, typical tissue sources comprise spleen, thymus, tissue biopsy, tumor, afferent lymph, lymph nodes, skin, GALT, bone marrow, apheresis or leukapheresis product, and/or peripheral blood. In certain embodiments, apheresis product, bone marrow and peripheral blood are preferred sources. Fetal tissue, fetal or umbilical cord blood, which is also rich in growth factors may also be used as a source of blood for obtaining precursor APC. Exemplary precursor cells may be, but are not limited to, embryonic stem cells, CD34⁺ cells, monocyte progenitors, monocytes, and pre-B cells. In another embodiment, cells or cell lines which would be likely to de-differentiate may be used as a source of precursor cells.

Further, according to one aspect of the present invention, precursor cells comprise monocytes or CD34⁺ cells.

In one aspect of the present invention, the starting material for producing immature APC and mature APC is an apheresis or leukapheresis product. Cells are collected using apheresis procedures known in the art. See, for example, Bishop et al., Blood 83(2):610-16, 1994. Briefly, cells are collected using conventional devices, for example, a Haemonetics Model V50 apheresis device (Haemonetics, Braintree, Mass.). Apheresis product typically contains lymphocytes, including T cells, monocytes, granulocytes, B cells, other nucleated white blood cells, red blood cells (RBC), and platelets. In one embodiment, the cells collected by apheresis may be washed to remove the plasma fraction and to place the cells in an appropriate buffer or media for subsequent processing steps. In another embodiment of the invention, the cells are washed with PBS. In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Gambro BCT, Lakewood, Colo.) according to the manufacturer's instructions. After washing, the cells may be resuspended in a variety of biocompatible buffers, such as, for example, Ca⁺⁺/Mg⁺⁺-free PBS. Alternatively, the undesirable components of the apheresis sample may be removed and the cells directly resuspended in culture media.

In an alternative embodiment, CD34⁺ cells can be obtained from freshly isolated bone marrow or from an apheresis product in which mononuclear cells are already enriched. The mononuclear cells can be enriched by means of density centrifugation both when CD34⁺ cells are isolated directly from the blood and when CD34⁺ cells have been isolated from an apheresis product. While a density centrifugation is preferred, it is not required. If CD34⁺ cells have been isolated directly from the blood (heparinized blood samples), lysis of the erythrocytes may suffice and this may be followed, at the next purification step, by an affinity column or another enrichment step. If CD34⁺ cells are isolated directly from the apheresis product, these cells may be added to an affinity column after only one washing and without FICOLL separation. Enrichment using FICOLL gradients can be omitted, in particular, when relatively large quantities of CD34⁺ cells are already present, as can be the case, for example, in association with high-dose chemotherapy. CD34⁺ cells may also be enriched by negative selection with a combination of antibodies directed to surface markers unique to the negatively selected cells. A preferred method is cell sorting and/or selection via negative magnetic immunoadherence or flow cytometry that uses a cocktail of mAb directed to cell surface markers present on the cells to be negatively selected

The mononuclear cells may be subjected to further treatment in order to enrich those cells which possess the CD34 surface antigen. Berenson et al. described the CD34 antigen in the publication “Engraftment After Infusion of CD34⁺ Marrow Cells in Patients With Breast Cancer or Neuroblastoma” (Blood 77(8):1717-22, 1991). These cells can be enriched by incubating the cells with a monoclonal antibody which is specific for the CD34 antigen, with the antibody conjugated to, for example, biotin. mAb of this kind can be obtained commercially, for example from Dianova, Coulter, or Becton Dickinson. The cells which have been treated with the monoclonal antibody are loaded on to immunoaffinity columns, e.g., avidin immunoaffinity columns, where the avidin binds the mAb and consequently also the CD34⁺ cells which are bound to the antibodies. The absorbed cells, possessing the CD34 surface antigen, are removed from the immunoaffinity column and introduced into a suitable medium.

Likewise, the mAb which are specific for the CD34 antigen could be bound directly to a solid phase (for example small beads, etc.) in order to fix the CD34⁺ cells and remove them from the mixture.

In addition, it is possible to enrich the CD34⁺ cells using a fluorescence-activated cell sorter, which can be obtained commercially, for example from Becton Dickinson or Cytomation. In this procedure, mobilized peripheral blood progenitor cells are reacted with an anti-CD34 antibody which possesses a fluorochrome label. Using the fluorescence-activated cell sorter, it is possible to separate the cells in order to obtain the CD34⁺ cells. Highly purified cells can be obtained in this way. CD34⁺ cells may also be enriched by negative selection with a combination of antibodies directed to surface markers unique to the negatively selected cells. A preferred method is cell sorting and/or selection via negative magnetic immunoadherence or flow cytometry that uses a cocktail of mAb directed to cell surface markers present on the cells to be negatively selected. Another possibility would be to separate the CD34⁺ cells by using magnetic beads which can be obtained commercially from Dynal, Baxter, Miltenyi and other firms.

The enriched CD34⁺ cells may then be cultured in a suitable culture medium. An example of such a medium is supplemented RPMI 1640 medium which contains 10% human AB serum. The culture medium can also contain heparinized autologous plasma, for example at a concentration of approximately 1%. RPMI 1640 medium which is supplemented with 2 mM L-glutamine, 50 μM β-mercaptoethanol, 1 mM sodium pyruvate, 50 μg/ml streptomycin, 50 U/ml penicillin, MEM vitamins and 10% human AB serum, or FBS may be used as the culture medium. Other media that may be used include X-Vivo 15, X-Vivo 20, and AIM V, supplemented with appropriate sera, vitamins, and amino acids.

In one aspect of the present invention, the source of precursor APCs are embryonic stem (ES) cells. ES cells may be isolated and cultured as described in U.S. Pat. No. 6,200,806 (see also Thomson et al., Science 282:1145-47, 1998). Briefly, a preferable medium for isolation of embryonic stem cells is “ES medium.” ES medium consists of 80% Dulbecco's modified Eagle's medium (DMEM; no pyruvate, high glucose formulation, Gibco BRL), with 20% serum, 0.1 mM β-mercaptoethanol (Sigma), 1% non-essential amino acid stock (Gibco BRL). Tissue culture dishes are preferably treated with 0.1% gelatin (type I; Sigma).

According to certain methods of the invention, any tissue that contains a source of progenitor cells may be used. The tissue sources may be treated prior to culturing to enrich the proportion of precursor APC relative to other cell types. Such pretreatment may also remove cells that may compete with the proliferation of precursor cells or inhibit their proliferation or survival. Pretreatment may also be used to make the tissue source more suitable for in vitro culture, for example grinding and/or enzymatic digestion. The method of treatment will likely be tissue-specific depending on the particular tissue source. For example, spleen or bone marrow, if used as a tissue source, would first be treated so as to obtain single cells followed by suitable cell separation techniques to separate leukocytes from other cell types. Treatment of blood may involve cell separation techniques to separate leukocytes from other cell types including RBC. Removal of RBCs may be accomplished by standard methods known to those skilled in the art.

In one form of pretreatment, cells that compete and mask the proliferation of precursor APC are rendered inoperative. Such pretreatment comprises killing cells expressing antigens which are not expressed on precursor cells by exposing the cell source to antibodies specific for antigens not present on precursor cells in a medium comprising complement. Another form of pretreatment to remove undesirable cells suitable for use with this invention is adsorbing the undesirable precursor cells or their progenitor onto a solid support using antibodies specific for antigens expressed on the undesirable cells. Several methods of adsorbing cells to solid supports of various types are known to those skilled in the art and are suitable for use with this invention. For example, undesirable cells may be removed by “panning” using a plastic surface such as a petri dish. Alternatively, other methods which are among those suitable include adsorbing cells onto magnetic beads to be separated by a magnetic force; or immunobeads to be separated by gravity. Non-adsorbed cells containing an increased proportion of precursor cells may then be separated from the cells adsorbed to the solid support by known means including panning. These pretreatment steps serve a dual purpose: they destroy or revive the precursors of non-APC cells in the culture while increasing the proportion of APC precursors competing for nutrients in the culture. Precursor cells may also be separated by bouyant density centrifugation or by elutriation, using, for example, a Beckman J6ME centrifuge equipped with a J5.0 rotor and a 40 ml elutriation chamber.

Positive selection may also be used to increase the proportion of desired APC precursors in the culture. Numerous immunoselection methods known to skilled artisans may be used. Such techniques are described, for example, in Current Protocols in Immunology, John Wiley & Sons, New York, N.Y. Cell surface markers that may be used to positively select APC precursors include, but are not limited to CD14, CD1a, CD40, CD86, CD54, and MHC class II molecules. According to one embodiment, precursor APC populations may be isolated from blood preparations by a variety of methodologies, including anti-CD14 coated beads or columns. APC may also be enriched by negative selection with a combination of antibodies directed to surface markers unique to the negatively selected cells. A preferred method is cell sorting and/or selection via negative magnetic immunoadherence or flow cytometry that uses a cocktail of mAb directed to cell surface markers present on the cells to be negatively selected.

In one embodiment of the present invention, isolation of precursor APC is performed by preincubating with PBMC separated from whole blood or apheresed peripheral blood with one or more varieties of irrelevant or non-antibody coupled paramagnetic particles (approx. 1 vial of beads or 4×10⁹ beads to one batch of cells (typically from about 5×10⁸ to about 2×10¹⁰ cells) for about 30 minutes to 2 hours at 22 to 37° C., followed by magnetic removal of cells which have attached to or engulfed the paramagnetic particles. Such separation can be performed using standard methods available in the art. For example, any magnetic separation methodology may be used including a variety of which are commercially available (e.g., DYNAL® Magnetic Particle Concentrator (DYNAL MPC®)). Assurance of isolation can be monitored by a variety of methodologies known to those of ordinary skill in the art, including flow cytometric analysis of CD14⁺ cells, before and after said isolation.

When blood is used as a tissue source, blood leukocytes may be obtained using conventional methods that maintain their viability. According to one aspect of the invention, blood is diluted into medium (preferably RPMI) that may or may not contain heparin (about 100 U/ml) or other suitable anticoagulant. The volume of blood to medium is about 1 to 1. Cells are concentrated by centrifugation of the blood in medium at about 1000 rpm (150 g) at 4° C. Platelets and RBC are depleted by resuspending the cells in any number of solutions known in the art that will lyse erythrocytes, for example ammonium chloride. For example, the mixture may be medium and ammonium chloride (at a final concentration of about 0.839%) at about 1:1 by volume. Cells may be concentrated by centrifugation and washed about 2 more times in the desired solution until a population of leukocytes, substantially free of platelets and RBC, is obtained. Any isotonic solution commonly used in tissue culture may be used as the medium for separating blood leukocytes from platelets and RBC. Examples of such isotonic solutions are PBS, Hanks balanced salt solution, or complete growth media including for example RPMI 1640, DMEM, MEM, HAMS F-12, X-Vivo 15, or X-Vivo 20. Precursor cells may also be purified by elutriation, using, for example, a Beckman J6ME centrifuge equipped with a J5.0 rotor and a 40 ml elutriation chamber.

Those of ordinary skill in the art will readily appreciate that the cell separation and culture methodologies described herein, may be carried out in a variety of environments (i.e., containers). Examples include various bags (e.g., Lifecell culture bags), flasks, roller bottles, bioreactors, (e.g., CellCube (Corning Science Products) or CELL-PHARM, (CD-Medical, Inc. of Hialeah, Fla.)), petri dishes and multi-well containing plates made for use in tissue culture, or any container capable of holding cells, preferably in a sterile environment. In one embodiment of the present invention a bioreactor is also useful. For example, several manufacturers currently manufacture devices that can be used to grow cells and be used in combination with the methods of the present invention. See for example, Celdyne Corp., Houston, Tex.; Unisyn Technologies, Hopkinton, Mass.; Synthecon, Inc. Houston, Tex.; Aastrom Biosciences, Inc. Ann Arbor, Mich.; Wave Biotech LLC, Bedminster, N.J. Further, patents covering such bioreactors include U.S. Pat. Nos. 6,096,532; 5,985,653; 5,888,807; 5,190,878, which are incorporated herein by reference.

In certain aspects of the present invention, it is not required that the APC or the activated T cells or supernatant therefrom described herein be derived from an autologous source. Thus, the APC and activated T cells or supernatant therefrom can be obtained from a matched or unmatched donor, or from a cell line, a T cell line, or other cells grown in vitro. Methods for matching haplotypes are known in the art. Furthermore, the APC and activated T cells or supernatant therefrom may be obtained from a xenogeneic source, for example, mouse, rat, non-human primate, and porcine cells may be used.

Methods of Generating Immature pAPC

Precursor cells obtained from treatment of the tissue source may be cultured to form a primary culture in an appropriate culture container or vessel in a culture medium supplemented with the appropriate cytokine or cytokines. According to the present invention, the appropriate culture container or vessel may be any container with tissue culture compatible surface. Examples include various bags (e.g., Lifecell culture bags), flasks, roller bottles, petri dishes and multi-well containing plates made for use in tissue culture. Surfaces treated with a substance, for example collagen or poly-L-lysine, or antibodies specific for a particular cell type to promote cell adhesion may also be used provided they allow for the differential attachment of cells as described below. Surfaces may be also be chemically treated, for example by ionization. Cells are plated at an initial cell density from about 10⁵ to 10⁷ cells/cm². In one aspect, cells are plated at 10⁶ cells/cm².

The growth medium for the cells at each step of the method of the invention should allow for the survival and differentiation of the precursor APC into immature and then mature APC. Any growth medium typically used to culture cells may be used according to the method of the invention provided the medium is supplemented with the appropriate cytokines. According to the present invention, the cytokines may be, but are not limited to, GM-CSF and interleukin 4 (IL-4), or IL-13. Other exemplary cytokines and growth factors that may be added to the growth medium include but are not limited to interleukin 1α (IL-1α) and β (IL-1β), tumor necrosis factor alpha (TNF-α), interleukin 3 (IL-3), macrophage colony stimulating factor (M-CSF), granulocyte colony-stimulating factor (G-CSF), stem cell factor (SCF), interleukin 6 (IL-6), and Flt3-L. Preferred media include RPMI 1640, AIM-V, DMEM, MEM, α-MEM, F-12, X-Vivo 15, and X-Vivo 20, with added amino acids and vitamins, either serum-free or supplemented with an appropriate amount of serum (or plasma) or a defined set of hormones, and an amount of cytokine(s) sufficient to promote the differentiation of precursor cells to the immature state. In one aspect, media may include lipids and/or sources of protein. RPMI 1640 supplemented with 1-5% human AB serum and a mixture of GM-CSF and IL-4 or IL-13 is preferred, although other mixtures of cytokines may also be used. Cells may also be adapted to grow in other sera, such as fetal calf (bovine) serum (FCS/FBS), at other concentrations of serum, or in serum-free media. For example, serum-free medium supplemented with hormones is also suitable for culturing the APC precursors. Media may, but does not necessarily, contain antibiotics to minimize growth of bacteria in the cultures. Penicillin, streptomycin or gentamicin or combinations containing them are preferred. The medium, or a portion of the medium, in which the cells are cultured should be periodically replenished to provide fresh nutrients including GM-CSF, IL-4, IL-13, and/or other cytokines.

In one embodiment, the media may contain chemokines including, but not limited to, IFN-γ inducible protein-10 (gIP-10), interleukin-8 (IL-8), platelet factor-4 (PF4), neutrophil activating protein (NAP-2), GRO-α, GRO-β, GRO-γ, neutrophil-activating peptide (ENA-78), granulocyte chemoattractant protein-2 (GCP-2), and stromal cell-derived factor-1 (SDF-1, or pre-B cell stimulatory factor (PBSF)); and/or a β(CC) chemokine selected from the group consisting of: regulated on activation, normal T expressed and secreted (RANTES), macrophage inflammatory protein-1α (MIP-1α), macrophage inflammatory protein-1β (MIP-1β), monocyte chemotactic protein-1 (MCP-1), monocyte chemotactic protein-2 (MCP-2), monocyte chemotactic protein-3 (MCP-3), monocyte chemotactic protein-4 (MCP-4), macrophage inflammatory protein-1γ (MIP-1γ), macrophage inflammatory protein-3α (MIP-3α), macrophage inflammatory protein-3β (MIP-3β), eotaxin, Exodus, and 1-309; and/or the γ(C) chemokine, lymphotactin. Chemokines may be used in varying concentrations that range from 1 ng/ml to 10 μg/ml, depending on the culture conditions.

According to one embodiment of the present invention, GM-CSF may be used in growth medium at a concentration of between about 10 to 200 ng/ml, or any integer value in between. Typically, a concentration of 100 ng/ml is used. Cells from bone marrow require higher concentrations of GM-CSF because of the presence of proliferating granulocytes which compete for the available GM-CSF, therefore, doses between about 50 to 400 ng/ml are preferred for cultures of cells obtained from marrow, unless such populations are pretreated to remove granulocytes.

GM-CSF may be isolated from natural sources, produced using recombinant DNA techniques or prepared by chemical synthesis. As used herein, GM-CSF includes GM-CSF produced by any method and from any species. “GM-CSF” is defined herein as any bioactive analog, fragment or derivative of the naturally occurring (native) GM-CSF. Such fragments or derivative forms of GM-CSF should also promote the proliferation in culture of APC precursors. In addition, GM-CSF peptides having biologic activity can be identified by their ability to bind GM-CSF receptors on appropriate cell types.

It may be desirable to include additional cytokines in the culture medium in addition to GM-CSF to further increase the yield of immature APC. Such cytokines include but are not limited to, G-CSF, M-CSF, IL-1α, IL-1β, IL-3, IL-4, IL6, IL-13, TNF-α, SCF, and Flt3-L. Cytokines are used in amounts which are effective in increasing the proportion of immature APC present in the culture either by enhancing proliferation or survival of immature APC precursors. In certain aspects, cytokines are present in the following concentrations: IL-1α and β, 1 to 100 U/ml; TNF-α, 5-500 U/ml; IL-3, 25-500 U/ml; M-CSF, 100-1000 U/ml; G-CSF, 25-300 U/ml; SCF, 10-100 ng/ml; IL-4,4-100 ng/ml and IL-6, 10-100 ng/ml. In other aspects, concentrations of cytokines are: IL-1a, 50 U/ml; TNF-α, 50 U/ml; IL-3, 100 U/ml; M-CSF, 300 U/ml; and G-CSF, 100 U/ml. In related aspects, cytokines are human proteins. Preferred cytokines are produced from the human gene using recombinant techniques (rhu). TNFα at concentrations from about 10-50 U/ml may also be used to increase immature APC yields several fold.

In one embodiment, the primary cultures from the selected tissue source are allowed to incubate at about 37° C. under standard tissue culture conditions of humidity, CO₂, and pH until a population of cells has adhered to the substrate sufficiently to allow for the separation of nonadherent cells. Some immature APC in blood initially are nonadherent to plastic, particularly immature DC, in contrast to monocytes, so that the precursors can be separated after overnight culture. Monocytes and fibroblasts are believed to comprise the majority of adherent cells and usually adhere to the substrate within about 30 minutes to about 24 hours. In certain aspects, nonadherent cells are separated from adherent cells between about 1 to 16 hours. Nonadherent cells may be separated at about 1 to 2 hours. Any method which does not dislodge significant quantities of adherent cells may be used to separate the adherent from nonadherent cells. In certain aspects, the cells are dislodged by simple shaking or pipetting. Pipetting is most preferred.

Adherent cells comprising precursor APC (e.g., monocytes) isolated according to the methods of the invention are allowed to incubate at about 37° C. under standard tissue culture conditions of humidity, CO₂, and pH until a population of cells has reached an immature APC stage. In certain aspects, according to the present invention, adherent cells are allowed to incubate for a period of between 4 hours and 7 days. However, one of ordinary skill in the art will readily appreciate that incubation times and conditions may vary.

According to one aspect of the present invention, immature APC may be generated using supernatants from activated T cells (described in detail below). Supernatants from any source of T cells that are activated by any number of means described herein may be used to generate immature APC from precursor cells. For example, day 2-4, prefereably day 3, culture supernatant from T cells activated using anti-CD3×anti-CD28 magnetic bead stimulation may be collected and frozen for use at a later time, or may be used to culture precursor cells directly.

According to one aspect of the present invention, immature APC may be isolated directly from the nonadherent population of the selected tissue source described above.

In another aspect of the invention, immature APC may be obtained directly from peripheral blood using multiple density gradients generated from a single density gradient material as described in U.S. Pat. No. 6,121,044, and in more detail below. The isolation procedure may be completed in two days and is preferably performed entirely under serum-free conditions. The percent of immature APC in enriched, isolated fractions may be further increased by depleting contaminating cells using, for example, solid-phase antibody-based negative depletion. This procedure is based on a combination of density based separation of cell types and differentiation-induced changes in densities of cell types. An immature APC-containing sample, such as a sample from human peripheral blood (e.g., buffy coats) is diluted with a suitable buffer, such as Ca⁺⁺/Mg⁺⁺ free PBS, and layered onto a density gradient material or separation medium (preferably having a density of about 1.0770+/−0.0010 and an osmolarity of about 310+/−15) and centrifuged. Exemplary density gradient materials for this step include, but are not limited to, the silica-based Ficoll Equivalent Percoll (FEP), made from “PERCOLL” (Pharmacia LKB, Uppsala, Sweden), and Lymphoprep (Nycomed Laboratories, Oslo, Norway). The separations can be carried out either in any suitable tube, such as an ordinary 50 mL centrifugation tube.

The interface of the solutions in the centrifuged tubes contains peripheral blood mononuclear cells (PBMC), which are harvested, e.g., by pipeting the cells from the interface. The PBMC are then resuspended in a suitable buffer, such as D-PBS, and centrifuged to remove platelets (which remain in the supernatants). Platelet-depleted PBMC are again resuspended in a suitable buffer, such as D-PBS, and layered on a density gradient material or separation medium (preferably having a density of about 1.0650+/−0.0010 and an osmolarity of about 300+/−15) and centrifuged. An exemplary density gradient material for this step is the silica-based Monocyte Depletion Percoll (MDP).

The cells at the interface of the two solutions are primarily monocytes, while the concentrated cells are primarily lymphocytes. The monocyte (interface) fraction may be resuspended in a suitable culture medium, such as cold pooled human AB serum to which an equal volume of 80% AB serum 20% dimethyl sulfoxide (DMSO) is added dropwise, and frozen until needed.

The concentrated cells comprise a monocyte-depleted cell fraction containing peripheral blood lymphocytes and immature APC. These cells are harvested, washed, e.g., with D-PBS by centrifugation at room temperature, and resuspended in a suitable culture medium.

According to the methods of the present invention, a fraction enriched in immature APC may be obtained by (i) obtaining, from a human blood sample, a monocyte-depleted cell fraction containing peripheral blood lymphocytes and APC precursor cells, (ii) culturing the cell fraction in a serum-free medium for a period sufficient to produce a morphological change in APC precursor cells to cells having the morphology of a more mature APC, (iii) harvesting non-adherent cells produced by the culturing, and (iv) enriching the portion of APC in the harvested cells by density centrifugation, to obtain a fraction enriched in immature APC cells. Although the exemplified method achieves step (i) by density centrifugation, it will be understood by one of ordinary skill in the art that other approaches may be used to obtain such a monocyte-depleted cell fraction. Further, the isolation, enrichment and culture procedures described herein may be conveniently performed in a closed device/kit configuration. In one embodiment of the present invention, the process for preparing immature APC may comprise the following steps: a) In order to mobilize cells, GM-CSF, IL-4, or IL-13, either in combination or individually, is administered to the patient, with customary concentrations being administered. The effective amount of GM-CSF, IL-4, or IL-13 administered may be from 0.1 to 500 μg of GM-CSF, IL-4, or IL-13 per kilogram of body weight. More preferably, the effective amount administered is from 1 μg to 100 μg and most preferably from 5 to 50 μg of GM-CSF, IL-4, or IL-13 per kilogram of body weight; b) after a suitable period of time, approximately 50 to 100 ml of blood are removed; or the patient undergoes apheresis c) a Ficoll separation step can be carried out if the content of CD34⁺ cells is low; or the apheresis product is washed d) the erythrocytes can be lysed; e) a CD34⁻ isolation procedure can be carried out, which procedure, in one embodiment, is an immunoaffinity step. The immature APCs which are obtained in this way can be subjected to further treatment, such as maturation in the presence of growth factors and/or cytokines and/or activated T cells, as described herein, depending on the purpose, and then reintroduced into the patient. An apheresis for the purpose of enriching the stem cells may be used when relatively large quantities of APCs are required.

According to another aspect of the present invention, immature APC may be generated in vivo by the administration of Flt3-L (as described in U.S. Pat. Nos. 6,190,655 and 5,554,512) and/or soluble CD40L (described in U.S. Ser. No. 08/477,733, U.S. Ser. No. 08/484,624, U.S. Pat. No. 5,962,406) in conjunction with a pharmaceutically acceptable excipient. Flt3-L has been found to regulate the growth and differentiation of progenitor and stem cells. CD40L is a type II membrane polypeptide having an extracellular region at its C-terminus, a transmembrane region and an intracellular region at its N-terminus. Soluble CD40L comprises an extracellular region of CD40L or a fragment thereof. Flt3-L and/or CD40L can be administered alone or in sequential or concurrent combination with cytokines selected from the group listed above.

Flt3-L and/or CD40L can be formulated according to known methods used to prepare pharmaceutically useful compositions. Flt3-L and/or CD40L can be combined in admixture, either as the sole active material or with other known active materials, with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. in addition, such compositions can contain flt3-L and/or CD40L complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of flt3-L and/or CD40L. Flt3-L and/or CD40L can also be conjugated to antibodies against tissue-specific receptors, ligands or antigens, or coupled to ligands of tissue-specific receptors.

Flt3-L and/or CD40L can be administered topically, parenterally, or by inhalation. The term “parenteral” includes subcutaneous injections, intravenous, intramuscular, intracisternal injection, or infusion techniques. These compositions will typically contain an effective amount of the flt3-L and/or CD40L, alone or in combination with an effective amount of any other active material. Such dosages and desired drug concentrations contained in the compositions may vary depending upon many factors, including the intended use, mammal's body weight and age, and route of administration. Preliminary doses can be determined according to animal tests, and the scaling of dosages for human administration can be performed according to art-accepted practices. Keeping the above description in mind, typical dosages of Flt3-L and/or CD40L may range from about 10 μg per square meter to about 1000 μg per square meter. A preferred dose range is on the order of about 100 μg per square meter to about 300 μg per square meter.

Compositions comprising cytokines, chemokines, Flt3-L, and/or CD40L as described above will be administered at an effective amount. An “effective amount” means an amount capable of mobilizing or generating APC in vivo. It will be apparent to those of skill in the art that the effective amount of cytokine or chemokine will depend, inter alia, upon the patient, the dose, the administration schedule of the cytokine or chemokine, whether the cytokine or chemokine is administered alone or in conjunction with other therapeutic agents, the serum half-life of the composition, and the general health of the patient. The cytokine or chemokine is preferably administered in a composition including a pharmaceutically acceptable carrier. “Pharmaceutically acceptable carrier” means a carrier that does not cause any untoward effect in patients to whom it is administered. Such pharmaceutically acceptable carriers are well known in the art.

Positive selection may be used to isolate the immature APC generated either in vivo or in vitro as described herein. Numerous immunoselection methods known to skilled artisans may be used. Such techniques are described, for example, in Current Protocols in Immunology, John Wiley & Sons, New York. N.Y. Markers that may be useful for the positive selection of immature APC include, but are not limited to, CD1 a, CD40, CD86, CD54, MHC class II. In one embodiment, fluorescence activated cell sorting may also be used to isolate desired immature APC.

In one aspect of the present invention, negative selection of unwanted cells may be used to enrich the population of desired immature APC from a sample. Numerous immunoselection methods are known to those of skill in the art. Such techniques are described, for example, in Current Protocols in Immunology, John Wiley & Sons, New York. N.Y. A preferred method is cell sorting and/or selection via negative magnetic immunoadherence or flow cytometry that uses a cocktail of mAb directed to cell surface markers present on the cells negatively selected.

Phenotype and Function of Immature pAPC

Various techniques may be used to characterize the phenotype of cells present in tissue sources and cell cultures. These techniques may include analysis of morphology, detecting cell type specific antigens with mAb and cytometric analysis, identifying proliferating cells using tritiated thymidine autoradiography, assaying mixed leukocyte reactions, and demonstrating cell homing.

In certain embodiments of the present invention, immature APC, such as immature DC, may be CD14⁻ or CD14⁺ depending on the origin of the precursor cells. Immature DC may also express CD1a, CD40, CD86, CD54, and intermediate levels of MHC class II. Immature DC typically do not express CCR7 or CD83. The function of immature DC is to endocytose or phagocytose antigen. As the cells continue to mature, presentation of antigen increases.

T Cell Compositions

T cells can be obtained from a number of sources, including PBMC, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may also be obtained from a xenogeneic source, for example, from mouse, rat, non-human primate, and pig.

In certain embodiments of the present invention, any number of T cell lines available in the art, may be used. In certain embodiments of the present invention, T cells can be obtained from a unit of blood collected from a subject using any number of techniques known to the skilled artisan, such as FICOLL separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, RBC, and platelets. In one embodiment, the cells collected by apheresis or leukapheresis may be washed to remove the plasma fraction and to place the cells in an appropriate buffer or media for subsequent processing steps. In one embodiment of the invention, the cells are washed with PBS. In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, Baxter) according to the manufacturer's instructions. After washing, the cells may be resuspended in a variety of biocompatible buffers, such as, for example, Ca⁺⁺/Mg⁺⁺ free PBS. Alternatively, the undesirable components of the apheresis sample may be removed and the cells directly resuspended in culture media.

In another embodiment, T cells are isolated from peripheral blood lymphocytes by lysing the RBC, isolating and reserving the monocytes as described previously, or for example, by centrifugation through a PERCOLL™ gradient. A specific subpopulation of T cells, such as CD28⁺, CD4⁺, CD8⁺, CD45RA⁺, and CD45RO⁺T cells, can be further isolated by positive or negative selection techniques. For example, CD3⁺, CD28⁺T cells can be positively selected using CD3/CD28 conjugated magnetic beads (e.g., DYNABEADSO® M-450 CD3/CD28 T Cell Expander). For example, in one preferred embodiment, T-cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further emobdiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of mAb directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g. particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g. particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

Accordingly, in one embodiment, the invention uses paramagnetic particles of a size sufficient to be engulfed by phagocytotic monocytes, that are subsequently removed through magnetic separation. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments, the irrelevant beads include beads coated with sheep anti-mouse antibodies, goat anti-mouse antibodies, and human serum albumin.

Another method to prepare the T cells for stimulation is to freeze the cells after the washing step, which does not require the monocyte-removal step. Wishing not to be bound by theory, the freeze and subsequent thaw step provides a more uniform product by removing granulocytes and, to some extent, monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 10 per minute and stored in the vapor phase of a liquid nitrogen storage tank. Other methods of controlled freezing may be used as well as uncontrolled freezing immediately at −20° C. or in liquid nitrogen.

The activated T cells of the present invention are generated by cell surface moiety ligation that induces activation. The activated T cells are generated by activating a population of T cells and stimulating an accessory molecule on the surface of the T cells with a ligand which binds the accessory molecule, as described for example, in U.S. patent application number _____, entitled Simultaneous Stimulation and Concentration of Cells, filed on Apr. 26, 2002, U.S. patent application Ser. Nos. 08/253,694, 08/403,253, 08/435,816, 08/592,711, 09/183,055, 09/350,202, and 09/252,150, and U.S. Pat. Nos. 5,858,358 and 5,883,223, hereby incorporated by reference in their entirety.

Generally, T cell activation may be accomplished by cell surface moiety ligation, such as stimulating the T cell receptor (TCR)/CD3 complex or the CD2 surface protein. A number of anti-human CD3 mAb are commercially available, exemplary are, clone BC3 (XR-CD3; Fred Hutchinson Cancer Research Center, Seattle, Wash.), OKT3, prepared from hybridoma cells obtained from the American Type Culture Collection, and monoclonal antibody G19-4. Similarly, stimulatory forms of anti-CD2 antibodies are known and available. Stimulation through CD2 with anti-CD2 antibodies is typically accomplished using a combination of at least two different anti-CD2 antibodies. Stimulatory combinations of anti-CD2 antibodies that have been described include the following: the T11.3 antibody in combination with the T11.1 or T11.2 antibody (Meuer et al., Cell 36:897-906, 1984), and the 9.6 antibody (which recognizes the same epitope as T11.1) in combination with the 9-1 antibody (Yang et al., J. Immunol. 137:1097-1100,1986). Other antibodies that bind to the same epitopes as any of the above described antibodies can also be used. Additional antibodies, or combinations of antibodies, can be prepared and identified by standard techniques. Stimulation may also be achieved through contact with antigen, peptide, protein, peptide-MHC tetramers (see Altman et al., Science 274(5284):94-96,1996), superantigens (e.g., Staphylococcus enterotoxin A (SEA), Staphylococcus enterotoxin B (SEB), Toxic Shock Syndrome Toxin 1 (TSST-1)), endotoxin, or through a variety of mitogens, including but not limited to, phytohemagglutinin (PHA), phorbol myristate acetate (PMA) and ionomycin, lipopolysaccharide (LPS), T cell mitogen, and IL-2.

To further activate a population of T cells, a co-stimulatory or accessory molecule on the surface of the T cells, such as CD28, is stimulated with a ligand that binds the accessory molecule. Accordingly, one of ordinary skill in the art will recognize that any agent, including an anti-CD28 antibody or fragment thereof capable of cross-linking the CD28 molecule, or a natural ligand for CD28 can be used to stimulate T cells. Exemplary anti-CD28 antibodies or fragments thereof useful in the context of the present invention include monoclonal antibody 9.3 (IgG2ₐ) (Bristol-Myers Squibb, Princeton, N.J.), monoclonal antibody KOLT-2 (IgG1), 15E8 (IgG1), 248.23.2 (IgM), clone B-T3 (XR-CD28; Diaclone, Besancon, France) and EX5.3D10 (IgG2a) (ATCC HB11373). Exemplary natural ligands include the B7 family of proteins, such as B7-1 (CD80) and B7-2 (CD86) (Freedman et al., J. Immunol. 137:3260-67,1987; Freeman et al., J. Immunol. 143:2714-22,1989; Freeman et al., J. Exp. Med. 174:625-31,1991; Freeman et al., Science 262:909-11, 1993; Azuma et al., Nature 366:76-79, 1993; Freeman et al., J. Exp. Med. 178:2185-92, 1993).

In addition, binding homologues of a natural ligand, whether native or synthesized by chemical or recombinant techniques, can also be used in accordance with the present invention. Other agents may include natural and synthetic ligands. Agents may include, but are not limited to, other antibodies or fragments thereof, a peptide, polypeptide, growth factor, cytokine, chemokine, glycopeptide, soluble receptor, steroid, hormone, mitogen, such as PHA, or other superantigens.

Methods of Generating pAPC

The mature APC of the invention are produced by exposing activated T cells, with or without antigen, in vivo or in vitro, to the immature APC prepared according to the method of the invention. In one embodiment, immature APC are plated in culture dishes and exposed to antigen in a sufficient amount and for a sufficient period of time to allow the antigen to bind and/or be taken up by the APC. In certain aspects, activated T cells and antigen are exposed to the immature APC for a period of time between 24 hours and 4 days. The amount and time necessary to achieve binding and uptake of the antigen by the APC may be determined by immunoassay or binding assay. Other methods known to those of skill in the art may be used to detect the presence of antigen in the context of MHC on the APC following their exposure to antigen.

According to the present invention, the source of antigen may be, but is not limited to, protein, including glycoprotein, peptides, superantigens (e.g., SEA, SEB, TSST-1) antibody/antigen complexes, tumor lysate, non-soluble cell debris, apoptotic bodies, necrotic cells, whole tumor cells from a tumor or a cell line that have been treated such that they are unable to continue dividing, allogeneic cells that have been treated such that they are unable to continue dividing, irradiated tumor cells, irradiated allogeneic cells, natural or synthetic complex carbohydrates, lipoproteins, LPS, RNA or a translation product of said RNA, and DNA or a polypeptide encoded by said DNA. Non-transformed cells are typically irradiated with gamma rays in the range of about 3000 to 3600 rads, more preferably at about 3300 rads. Lymphoblastoid or tumor cell lines are typically irradiated with gamma rays in the range of about 6000 to 10,000 rads, more preferably at about 8000 rads. Necrotic and apoptotic cells may be generated by physical, chemical, or biological means. Necrotic cells are typically generated by freeze-thawing, while apoptotic cells are generated using UV irradiation. UV and gamma irradiation, and freeze-thawing procedures are well known in the art and are described, for example, in Current Protocols in Molecular Biology or Current Protocols in Immunology, John Wiley & Sons, New York, N.Y.

The APC of the present invention may be loaded with antigen through genetic modification. Genetic modification may comprise RNA or DNA transfection using any number of techniques known in the art, for example electroporation (using e.g., the Gene Pulser II, BioRad, Richmond, Calif.), various cationic lipids, (LIPOFECTAMINE™, Life Technologies, Carlsbad, Calif.), or other techniques such as calcium phosphate transfection as described in Current Protocols in Molecular Biology, John Wiley & Sons, New York, N.Y. For example, 5-50 μg of RNA or DNA in 500 μl of Opti-MEM can be mixed with a cationic lipid at a concentration of 10 to 100 μg, and incubated at room temperature for 20 to 30 minutes. Other suitable lipids include LIPOFECTIN™, LIPOFECTAMINE™. The resulting nucleic acid-lipid complex is then added to 1-3×10⁶ cells, preferably 2×10⁶, APC in a total volume of approximately 2 ml (e.g., in Opti-MEM), and incubated at 37° C. for 2 to 4 hours. The APC may also be transduced using viral transduction methodologies as described below.

Antigen source may also comprise non-transformed, transformed, transfected, or transduced cells or cell lines. Cells may be transformed, transfected, or transduced using any of a variety of expression or retroviral vectors known to those of ordinary skill in the art that may be employed to express recombinant antigens. Expression may also be achieved in any appropriate host cell that has been transformed, transfected, or transduced with an expression or retroviral vector containing a DNA molecule encoding recombinant antigen(s). Any number of transfection, transformation, and transduction protocols known to those in the art may be used, for example those outlined in Current Protocols in Molecular Biology, John Wiley & Sons, New York, N.Y., or in numerous kits available commercially (e.g., Invitrogen Life Technologies, Carlsbad, Calif.). In one embodiment of the present invention, recombinant vaccinia vectors and cells infected with said vaccinia vectors, may be used as a source of antigen. Recombinant antigen may include any number of defined tumor antigens described below.

According to certain methods of the invention, antigen may comprise defined tumor antigens such as the melanoma antigen Melan-A (also referred to as melanoma antigen recognized by T cells or MART-1), melanoma antigen-encoding genes 1, 2, and 3 (MAGE-1, -2, -3), melanoma GP100, carcinoembryonic antigen (CEA), the breast cancer angtigen, Her-2/Neu, serum prostate specific antigen (PSA), Wilm's Tumor (WT-1), mucin antigens, MUC-1, -2, -3, -4, and B cell lymphoma idiotypes.

In one aspect of the present invention, activated T cells (or supernatant therefrom), such as those described in U.S. patent application number ______ entitled Simultaneous Stimulation and Concentration of Cells, filed on Apr. 26, 2002, U.S. patent application Ser. Nos. 08/253,694, 08/403,253, 08/435,816, 08/592,711, 09/183,055, 09/350,202, and 09/252,150, and U.S. Pat. Nos. 5,858,358 and 5,883,223, hereby incorporated by reference in their entirety, may be added to the precursor APCs directly, either alone or in conjunction with cytokines, and used to mature pAPC from the precursor APC stage through the immature APC stage to the mature pAPC stage. Antigen may or may not be added at the immature APC stage. Activated T cells are typically added at a ratio from about 0.1 T cell/APC to about 20 T cells/APC, and all integer values within that range. Preferably, activated T cells are added at a ratio of 1 to about 10 T cells/APC. One of ordinary skill in the art will appreciate that optimal ratios may be determined by techniques known in the art.

In one embodiment of the present invention, activated T cells, are added at the same time or after addition of antigen.

According to one aspect of the present invention, mature APC may be generated using only supernatants from activated T cells. Supernatants from any source of T cells that are activated by any number of means described herein may be used to generate immature APC from precursor cells. For example, day 1 to 4, prefereably day 2 to 3, culture supernatant from T cells activated using anti-CD3×anti-CD28 magnetic bead stimulation may be collected and frozen for use at a later time, or may be used to culture precursor cells directly, or may be added to immature APC with or without antigen.

In one embodiment of the present invention, mature pAPC are generated in vivo by administration of activated T cells either alone, in conjunction with or following administration of cytokines or chemokines, including but not limited to, GM-CSF, IL-4, IL-13, Flt3-L, CD40L MIP1-α, and RANTES. In one embodiment of the present invention, APC are mobilized in vivo by administration of a pharmaceutical composition comprising an effective amount (i.e. an amount sufficient to mobilize APC) of one or more of the chemokines and cytokines listed previously, including but not limited to, IL-8, RANTES, MIP-1α, MIP-1β, MCP-1, lymphotactin, G-CSF, GM-CSF, IL-4, IL-13, Flt3-L, and CD40L. Preferably, the chemokines and cytokines have been purified. Methods for administering pharmaceutical compositions are described in the section entitled Pharmaceutical Compositions.

In one embodiment of the present invention, in vivo maturation of APC is accomplished by co-localization of activated T cells and APCs (either generated in vivo or in vitro) through the use of paramagnetic beads and application of a magnetic force inside or outside a target tissue (as described, for example, in U.S. Pat. No. 6,203,487, hereby incorporated by reference in its entirety). Briefly, activated T cells and maturing or mature APC are exposed to paramagnetic beads conjugated to appropriate surface markers either in vivo or in vitro or a combination of the two such that binding of the paramagnetic particle to the cells occurs. If carried out in vitro, a composition comprising cells bound to the paramagnetic particles and a pharmaceutically acceptible excipient is administered to a mammal. A magnet may be placed adjacent to a target tissue, i.e., an area of the body or a selected tissue or organ into which local cell delivery is desired. The magnet can be positioned superficial to the body surface or can be placed internal to the body surface using surgical or percutaneous methods inside or outside the target tissue for local delivery. The magnetic particles bound to cells are delivered either by direct injection into the selected tissue or to a remote site and allowed to passively circulate to the target site or are actively directed to the target site with a magnet or the targeting ligand.

Mature APC, according to the present invention, are characterized by the capacity to activate naive T cells. Further, mature APC may express CD40, CD54, CD80, CD83, CD86, CCR7, ICAM-1, CD1a, and high levels of MHC class II, as measured by mAb staining and flow cytometric analysis. In one aspect of the present invention, the APC lose expression of CD14 as they mature.

The phenotypic properties of cell populations of the present invention can be monitored by a variety of methods including, microscopy, in situ hybridization, in situ polymerase chain reaction (PCR), standard flow cytometry methods, enzyme-linked immunosorbent assay (ELISA) and other methods known by those skilled in the art.

In one aspect of the present invention, the APC at any stage of maturation may be genetically modified using any number of methods known in the art. The APC may be transfected using numerous RNA or DNA expression vectors known to those of ordinary skill in the art. Genetic modification may comprise RNA or DNA transfection using any number of techniques known in the art, for example electroporation (using e.g., the Gene Pulser II, BioRad, Richmond, Calif.), various cationic lipids, (LIPOFECTAMINE™, Life Technologies, Carlsbad, Calif.), or other techniques such as calcium phosphate transfection as described in Current Protocols in Molecular Biology, John Wiley & Sons, New York, N.Y. For example, 5-50 μg of RNA or DNA in 500 μl of Opti-MEM can be mixed with a cationic lipid at a concentration of 10 to 100 μg, and incubated at room temperature for 20 to 30 minutes. Other suitable lipids include LIPOFECTIN™, LIPOFECTAMINE™. The resulting nucleic acid-lipid complex is then added to 1-3×10⁶ cells, preferably 2×10⁶, APC in a total volume of approximately 2 ml (e.g., in Opti-MEM), and incubated at 37° C. for 2 to 4 hours. The APC may also be transduced using viral transduction methodologies as described below The APC may alternatively be genetically modified using retroviral transduction technologies. In one aspect of the invention, the retroviral vector may be an amphotropic retroviral vector, preferably a vector characterized in that it has a long terminal repeat sequence (LTR). e.g., a retroviral vector derived from the Moloney murine leukemia virus (MoMLV), myeloproliferative sarcoma virus (MPSV), murine embryonic stem cell virus (MESV). murine stem cell virus (MSCV), spleen focus forming virus (SFFV), or adeno-associated virus (AAV). Most retroviral vectors are derived from murine retroviruses. Retroviruses adaptable for use in accordance with the present invention can, however, be derived from any avian or mammalian cell source. These retroviruses are preferably amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman, BioTechniques 7:980-90,1989; Miller, A. D., Human Gene Therapy 1:5-14,1990; Scarpa et al., Virology 180:849-52,1991; Burns et al., Proc. Natl. Acad. Sci. USA 90:8033-37,1993; and Boris-Lawrie and Temin, Cur. Opin. Genet. Develop. 3:102-09, 1993.

In another aspect of the present invention, mature APC are isolated from the activated T cells by positive or negative selection methods described herein. Likewise, the activated T cells may be isolated from the mature APC by positive or negative selection methods.

Pharmaceutical Compositions

An additional aspect of the present invention provides a population or composition of maturing and/or mature pAPC. In a related embodiment, the present invention provides a population or composition of maturing and/or mature pAPC and/or activated T cells.

The present invention further provides a pharmaceutical composition comprising the maturing and/or mature APC and a pharmaceutically acceptable carrier. Compositions of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, PBS and the like; carbohydrates such as glucose, mannose, sucrose or dextrans, mannitol; proteins; polypeptides or amino acids such as glycine; antioxidants; chelating agents such as ethylenediaminetetraacetic acid (EDTA) or glutathione; adjuvants (e.g., aluminum hydroxide); and preservatives. Compositions of the present invention are, in certain aspects, formulated for intravenous administration.

A related embodiment of the present invention further provides a pharmaceutical composition comprising the maturing and/or mature APC, activated T cells, and a pharmaceutically acceptable carrier. The pharmaceutically acceptable carrier should be sterilized by techniques known to those skilled in the art.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The present invention also provides methods for preventing, inhibiting, or reducing the presence of a cancer or malignant cells in an animal, which comprise administering to an animal an anti-cancer effective amount of the subject mature and/or maturing APC with or without activated T cells.

The cancers contemplated by the present invention, against which the immune response is induced, or which is to be prevented, inhibited, or reduced in presence, may include but are not limited to melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, pancreatic cancer, esophageal cancer, brain cancer, lung cancer, ovarian cancer, cervical cancer, multiple myeloma, hepatocellular carcinoma, nasopharyngeal carcinoma, ALL, AML, CML, CLL, and other neoplasms known in the art.

Alternatively, compositions as described herein can be used to induce or enhance responsiveness to pathogenic organisms, such as viruses, (e.g., single stranded RNA viruses, single stranded DNA viruses, double-stranded DNA viruses, HIV, hepatitis A, B, and C virus, HSV, CMV, EBV, HPV), parasites (e.g., protozoan and metazoan pathogens such as Plasmodia species, Leishmania species, Schistosoma species, Trypanosoma species), bacteria (e.g., Mycobacteria, Salmonella, Streptococci, E. coli, Staphylococci), fungi (e.g., Candida species, Aspergillus species) and Pneumocystis carinii.

The immune response induced in the animal by administering the subject compositions of the present invention may include cellular immune responses mediated by cytotoxic T cells, capable of killing tumor and infected cells, and helper T cell responses. Humoral immune responses, mediated primarily by B cells that produce antibodies following activation by helper T cells, may also be induced. A variety of techniques may be used for analyzing the type of immune responses induced by the compositions of the present invention, which are well described in the art; e.g., Coligan et al., Current Protocols in Immunology, John Wiley & Sons Inc., 1994.

When “an immunologically effective amount,” “an anti-tumor effective amount,” “an tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient. It can generally be stated that a pharmaceutical composition comprising the subject maturing and/or mature pAPC with or without activated T cells, may be administered at a dosage of 1 to 10⁷ APC/kg body weight, preferably 10⁵ to 10⁶ APC/kg body weight, including all integer values within those ranges. APC compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

Typically, in adoptive immunotherapy studies, activated T cells are administered approximately at 2×10⁹ to 2×10¹¹ cells to the patient. (See, e.g., U.S. Pat. No. 5,057,423). In some aspects of the present invention, particularly in the use of allogeneic or xenogeneic cells, lower numbers of cells, in the range of 10⁶/kilogram (10⁶−10¹¹ per patient) may be administered. T cell compositions may be administered multiple times at dosages within these ranges. The maturing or mature APC-based method of therapy may be combined with other methods, such as direct administration of the activated T cells of the invention. The activated T cells and maturing and/or mature APC may be autologous or heterologous to the patient undergoing therapy. If desired, the treatment may also include administration of mitogens (e.g., PHA) or lymphokines, cytokines, and/or chemokines (e.g., GM-CSF, IL-4, IL-13, Flt3-L, RANTES, MIP1-α, etc.) as described herein to enhance induction of the immune response.

The administration of the subject pharmaceutical compositions may be carried out in any convenient manner, including by aerosol inhalation, injection, ingestion, transfusion, implantation or transplantation. The compositions of the present invention may be administered to a patient subcutaneously, intradermally, intramuscularly, by intravenous (i.v.) injection, or intraperitoneally. Preferably, the APC compositions of the present invention are administered to a patient by intradermal or subcutaneous injection. The T cell compositions of the present invention are preferably administered by i.v. injection. The compositions of maturing or mature APC or activated T cells may be injected directly into a tumor or lymph node.

In yet another embodiment, the pharmaceutical composition can be delivered in a controlled release system. In one embodiment, a pump may be used (see Langer, Science 249:1527-33,1990; Sefton, CRC Crit. Ref Biomed. Eng. 14:201, 1987; Buchwald et al., Surgery 88:507, 1980; Saudek et al., N. Engl. J. Med. 321:574,1989). In another embodiment, polymeric materials can be used (see Langer and Wise (eds.), Medical Applications of Controlled Release, CRC Pres., Boca Raton, Fla., 1974; Smolen and Ball (eds.), Controlled Drug Bioavailability, “Drug Product Design and Performance,” Wiley, New York, 1984; Ranger and Peppas, J. Macromol. Sci. Rev. Macromol. Chem. 23:61,1983; see also Levy et al., Science 228:190 1985; During et al., Ann. Neurol. 25:351, 1989; Howard et al., J. Neurosurg. 71:105,1989). In yet another embodiment, a controlled release system can be placed in proximity of the therapeutic target, thus requiring only a fraction of the systemic dose (see, e.g., Langer and Wise (eds.), Medical Applications of Controlled Release, CRC Pres., Boca Raton, Fla., 1984, vol. 2, pp. 115-138).

The maturing and/or mature APC and T cell compositions of the present invention may also be administered using any number of matrices. Matrices have been utilized for a number of years within the context of tissue engineering (see, e.g., Lanza, Langer, and Chick (eds.), Principles of Tissue Engineering, 1997). The present invention utilizes such matrices within the novel context of acting as an artificial lymphoid organ to support, maintain, or modulate the immune system, typically through modulation of T cells. Accordingly, the present invention can utilize those matrix compositions and formulations which have demonstrated utility in tissue engineering. Accordingly, the type of matrix that may be used in the compositions, devices and methods of the invention is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized, as such these patents are incorporated by reference in their entirety. Matrices comprise features commonly associated with being biocompatible when administered to a mammalian host. Matrices may be formed from both natural or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release of seeded cells or produced cytokine or other active agent. In certain embodiments, the matrix of the present invention is flexible and elastic, and may be described as a semisolid scaffold that is permeable to substances such as inorganic salts, aqueous fluids and dissolved gaseous agents including oxygen.

A matrix is used herein as an example of a biocompatible substance. However, the current invention is not limited to matrices and thus, wherever the term matrix or matrices appears these terms should be read to include devices and other substances which allow for cellular retention or cellular traversal, are biocompatible, and are capable of allowing traversal of macromolecules either directly through the substance such that the substance itself is a semi-permeable membrane or used in conjunction with a particular semi-permeable substance.

In one aspect of the present invention, maturing and/or mature APC may be fused with tumor cells to form hybrid cell compositions (herein referred to as “hybrid cells,” “dendritic/tumor cell fusions” or “APC/tumor cell fusions” (see e.g., Kugler et al., Nature Medicine 6(3):332-36, 2000; PCT patent application No. WO9630030). Single cell suspensions from primary tumor samples may be obtained using techniques known in the art. Any tumor cell line or cells derived from any cancer described herein may also be used. Tumor cells and maturing and/or mature APC may be, for example, resuspended in an appropriate concentration of glucose solution and subjected to electroporation using a Gene Pulser II electroporator (BioRad, Richmond, Calif.). Electrofusion may be accomplished by aligning the cells to form cell-cell conjugates at about 100V/cm for 5-10 seconds. In a second step, a pulse of about 1,200V/cm at 25 μF may be applied to fuse the aligned cells. One skilled in the art will readily recognize that optimization of electroporation conditions including electroporation buffers, voltage, capacitance, and decay times, may be necessary. Such fusion cell compositions may be used according to the present invention, either alone, or in conjunction with maturing and/or mature APC, and/or activated T cells to generate immune responses in vivo or in vitro.

In one embodiment of the present invention, maturing and/or mature APC may be used to generate antigen-specific T cells in vitro or in vivo. T cells may be stimulated with APC loaded with antigen as previously described. Such stimulation is performed under conditions and for a time sufficient to permit the generation of T cells that are specific for the antigen of interest. For example, T cells (5×10⁶ cells/ml) and antigen-loaded APC (2.5×10⁵ cells/ml) may be cultured in conventional media as described herein, supplemented with 5-10% serum, 1 mM sodium pyruvate, with or withour 100 lU/ml penicillin, with or without 100 ug/ml streptomycin, and 5×10⁻⁵ M β-mercaptoethanol in 96 well U-bottom plates at a ratio of 20:1. After 5 days, cells may be tested for antigen-specificity in a standard 4 hours chromium release assay. Antigen-specific T cells may be further expanded using techniques known in the art (as described in U.S. Pat. No. 5,827,642). Stimulation of T cells with anti-CD3/anti-CD28 magnetic bead as described in the Examples may be carried out following the stimulation with antigen-loaded APC to further increase expansion of the desired antigen-specific T cells.

All references referred to within the text are hereby incorporated by reference in their entirety. Moreover, all numerical ranges utilized herein explicitly include all integer values within the range and selection of specific numerical values within the range is contemplated depending on the particular use. Further, the following examples are offered by way of illustration, and not by way of limitation.

### Example 1

### T Cell Stimulation

In certain experiments described herein, the process referred to as XCELLERATE I™ was utilized. In brief, in this process, the XCELLERATED T cells are manufactured from a peripheral blood mononuclear cell (PBMC) apheresis product. After collection from the patient at the clinical site, the PBMC apheresis are washed and then incubated with “uncoated” DYNABEADS® M-450 Epoxy. During this time phagocytic cells such as monocytes ingest the beads. After the incubation, the cells and beads are processed over a MaxSep Magnetic Separator in order to remove the beads and any monocytic/phagocytic cells that are attached to the beads. Following this monocyte-depletion step, a volume containing a total of 5×10⁸ CD3⁺ T cells is taken and set-up with 1.5×10⁹ DYNABEADS® M-450 CD3/CD28 T Cell Expander to initiate the XCELLERATE™ process (approx. 3:1 beads to T cells). The mixture of cells and DYNABEADS® M-450 CD3/CD28 T Cell Expander are then incubated at 37° C., 5% CO₂ for approximately 8 days to generate XCELLERATED T cells for a first infusion. The remaining monocyte-depleted PBMC are cryopreserved until a second or further cell product expansion (approximately 21 days later) at which time they are thawed, washed and then a volume containing a total of 5×10⁸ CD3⁺ T cells is taken and set-up with 1.5×10⁹ DYNABEADS® M-450 CD3/CD28 T Cell Expander to initiate the XCELLERATE Process for a second infusion. During the incubation period of ≈8 days at 37° C., 5% CO₂, the CD3⁺ T cells activate and expand. The anti-CD3 mAb (clone BC3; XR-CD3) is obtained from the Fred Hutchinson Cancer Research Center, Seattle, Wash. and the anti-CD28 mAb (clone B-T3; XR-CD28) is obtained from Diaclone (Besancon, France).

With a modified process referred to as XCELLERATE II™ the process described above was utilized with some modifications in which no separate monocyte depletion step was utilized and in certain processes the cells were frozen prior to initial contact with beads and further concentration and stimulation were performed. In one version of this process T cells were obtained from the circulating blood of a donor or patient by apheresis. Components of an apheresis product typically include lymphocytes, monocytes, granulocytes, B cells, other nucleated cells (white blood cells), RBC, and platelets. A typical apheresis product contains 1-2×10¹⁰ nucleated cells. The cells are washed with calcium-free, magnesium-free PBS to remove plasma proteins and platelets. The washing step was performed by centrifuging the cells and removing the supernatant fluid, which is then replaced by PBS. The process was accomplished using a semi-automated “flow through” centrifuge (COBE 2991 System, Gambro BCT, Lakewood, Colo.). The cells are maintained in a closed system as they are processed.

The cells may be further processed by depleting the non-binding cells, including monocytes, (enriched for activated cells) and then continuing with the stimulation. Alternatively, the washed cells can be frozen, stored, and processed later, which is demonstrated herein to increase robustness of proliferation as well as depleting granulocytes. In one example, to freeze the cells, a 35 ml suspension of cells is placed in a 250 ml Cryocyte™ freezing bag (Baxter) along with 35 ml of the freezing solution. The 35 ml cell suspension typically contains 3.5×10⁹ to 5.0×10⁹ cells in PBS. An equal volume of freezing solution (20% DMSO and 8% human serum albumin in PBS) is added. The cells are at a final concentration of 50×10⁶ cells/ml. The Cryocyte bag may contain volumes in the range of 30-70 ml, and the cell concentration can range from 10 to 200×10⁶ cells/ml. Once the Cryocyte bag is filled with cells and freezing solution, the bag is placed in a controlled rate freezer and the cells are frozen at 1 C/minute down to −80° C. The frozen cells are then placed in a liquid nitrogen storage system until needed.

The cells are removed from the liquid nitrogen storage system and are thawed at 37° C. To remove DMSO, the thawed cells are then washed with calcium-free, magnesium-free PBS on the COBE 2991 System. The washed cells are then passed through an 80 micron mesh filter. The thawed cells, approximately 0.5×10⁹ CD3⁺ cells, are placed in a plastic 1L Lifecell bag that contains 100 ml of calcium-free, magnesium-free PBS. The PBS contains 1%-5% human serum. 1.5×10⁹ CD3×CD28 beads (Dynabeads M-450 CD3/CD28 T Cell Expander) are also placed in the bag with the cells (3:1 DYNABEADS M-450 CD3/CD28 T Cell Expander:CD3⁺ T cells). The beads and cells are mixed at room temperature at 1 rpm (end-over-end rotation) for about 30 minutes. The bag containing the beads and cells is placed on the MaxSep Magnetic Separator (Nexell Therapeutics, Irvine, Calif.). Between the bag and the MaxSep, a plastic spacer (approximately 6 mm thick) is placed. (To increase the magnetic strength the spacer can be removed.) The beads and any cells attached to beads are retained on the magnet while the PBS and unbound cells are pumped away.

The CD3×CD28 beads and concentrated cells bound to the beads are rinsed with cell culture media (1 liter containing X-Vivo 15, BioWhittaker; with 50 ml heat inactivated pooled human serum, 20 ml 1 M Hepes, 10 ml 200 mM L-glutamine with or without about 100,000 I.U. IL-2) into a 3L Lifecell culture bag. After transferring the CD3×CD28 beads and positively selected cells into the Lifecell bag, culture media is added until the bag contains 1000 ml. The bag containing the cells is placed in an incubator (37° C. and 5% CO₂) and cells are allowed to expand, splitting the cells as necessary.

T cell activation and proliferation was measured by harvesting cells after 3 days and 8 days in culture. Activation of T cells was assessed by measuring cell size, the level of cell surface marker expression, particularly the expression of CD25 and CD154 on day 3 of culture. On day 8 cells are allowed to flow under gravity (approx. 150 ml/min) over the MaxSep magnet to remove the magnetic particles and the cells are washed and concentrated using the COBE device noted above and resuspended in a balanced electrolyte solution suitable for intravenous administration, such as Plasma-Lyte A® (Baxter-Healthcare).

As described, the XCELLERATE I™ refers to conditions similar to that above, except that stimulation and concentration were not performed and monocyte depletion was performed prior to stimulation.

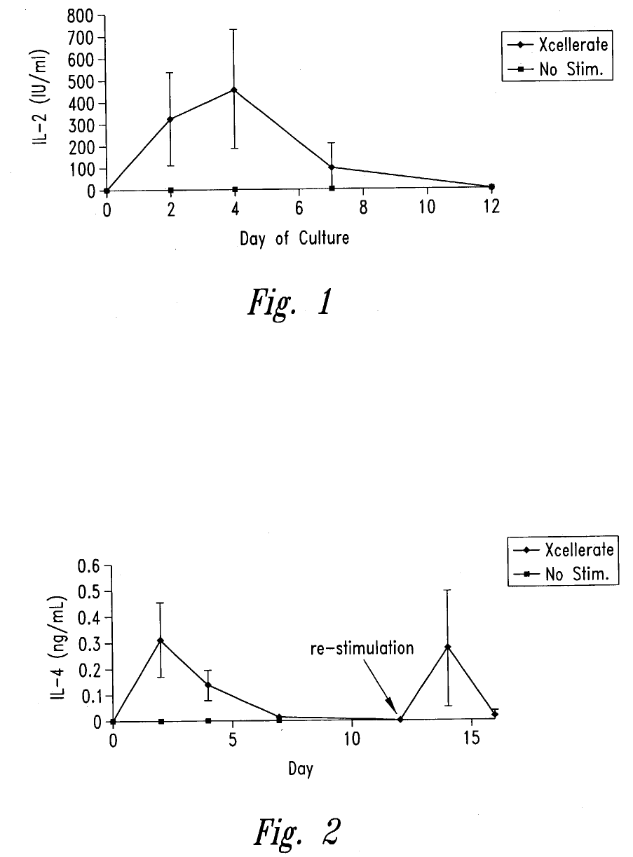

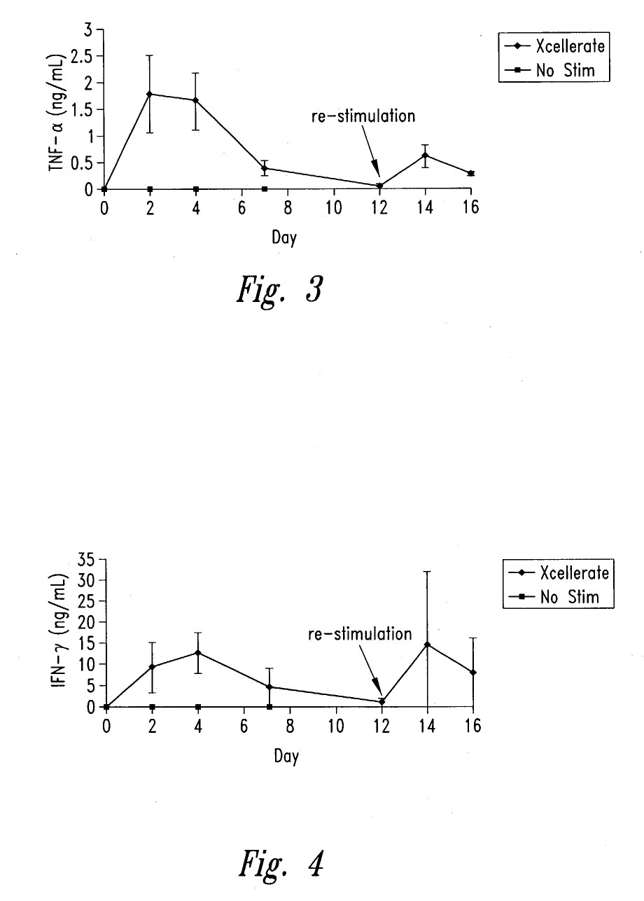

Monocyte-depleted PBMC from 4 donors were stimulated with CD3×CD28 coupled beads (Dynabeads M-450 CD3/CD28 T Cell Expander). The concentration of IL-2, IL-4, TNF-α and IFN-γ in the supernatant was determined on the days shown in FIGS. 1-4 by ELISA. Concentrations of IL-4, TNF-α and IFN-γ, were also measured following reseeding of the cells with new Dynabeads M-450 CD3/CD28 T Cell Expander on Day 12 (re-stimulation).

As shown in Table 1, Table 2, and Table 3, concentrations of IFN-γ, IL-4, and TNF-α, were measured by ELISA on various days during Xcellerate and Re-stimulation.

TABLE 1
Production of Interferon-y by T Cells on Day 3 of the Xcellerate Process
and on Day 2 of Re-stimulation of Xcellerate Activated T Cells

| | Xcellerate Process Day 3 | Re-stimulation Day 2 |
|---|---|---|
| | [IFN-γ] ng/mL | [IFN-γ] ng/mL |
| Average | 13.61 | 31.59 |
| Range | 7.99-27.11 | 10.8-95.5 |
| Standard Dev. | 5.64 | 22.98 |
| Median | 11.95 | 26.4 |
| N | 24 | 24 |

Phagocyte-depleted PMBC from 3 donors were stimulated with anti-CD3 & anti-CD28 coupled to Dynabeads M-450 Epoxy (Dynabeads CD3/CD28 T Cell Expander) (Xcellerate). The concentration of IFN-γ in the supernatant was determined on Day 2 by ELISA. On Day 12, cells were re-seeded with new anti-CD3 & anti-CD28 coupled Dynabeads M-450 Epoxy (re-stimulation) and the concentration of IFN-γ determined 2 days later.

TABLE 2
Production of IL-4 by T Cells on Day 3 of the Xcellerate Process
and on Day 2 of Re-stimulation of Xcellerate Activated T Cells

| | Xcellerate Process Day 3 | Re-stimulation Day 2 |
|---|---|---|
| | [IL-4] pg/ml | [IL-4] pg/ml |
| Average | 310 | 274 |
| Range | 170-460 | 50-500 |
| Standard Dev. | 143 | 224 |
| Median | 297 | 268 |
| N | 3 | 3 |

Phagocyte-depleted PMBC from 3 donors were stimulated with anti-CD3 & anti-CD28 coupled to Dynabeads M-450 Epoxy (Xcellerate). The concentration of IL-4 in the supernatant was determined on day 3 by ELISA. On Day 12, cells were re-seeded with new anti-CD3 & anti-CD28 coupled Dynabeads M-450 Epoxy (re-stimulation) and the concentration of IL-4 determined 2 days later.

TABLE 3
Production of TNF-α by T Cells on Day 2 & Day 4
of the Xcellerate Process and on Day 2 & Day 4 of
Re-stimulation of Xcellerate Activated T Cells

| | Day 2 | | Day 4 | |
|---|---|---|---|---|
| | Xcellerate | Re-stimulation | Xcellerate | Re-stimulation |
| | [TNF-α] | [TNF-α] | [TNF-α] | [TNF-α] |
| | ng/mL | ng/mL | ng/mL | ng/mL |
| Average | 1.710 | 0.594 | 1.635 | 0.252 |
| Range | 1.11-2.81 | 0.299-0.782 | 1.09-2.5 | 0.21-0.288 |
| Standard Dev. | 0.762 | 0.211 | 0.534 | 0.036 |
| Median | 1.460 | 0.647 | 1.55 | 0.255 |
| N | 4 | 4 | 4 | 4 |

Phagocyte-depleted PMBC from 4 donors were stimulated with anti-CD3 & anti-CD28 coupled to Dynabeads M-450 Epoxy. The concentration of TNF-α in the supernatant was determined on the days 2 & 4 by ELISA. On Day 12, cells were re-seeded with new anti-CD3 & anti-CD28 coupled Dynabeads M-450 Epoxy (re-stimulation) and the concentration of TNF-α determined 2 & 4 days later.

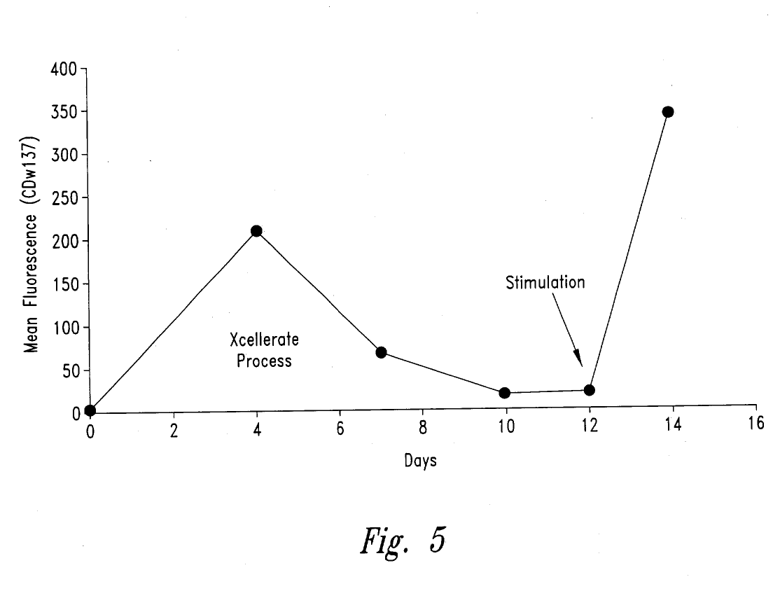

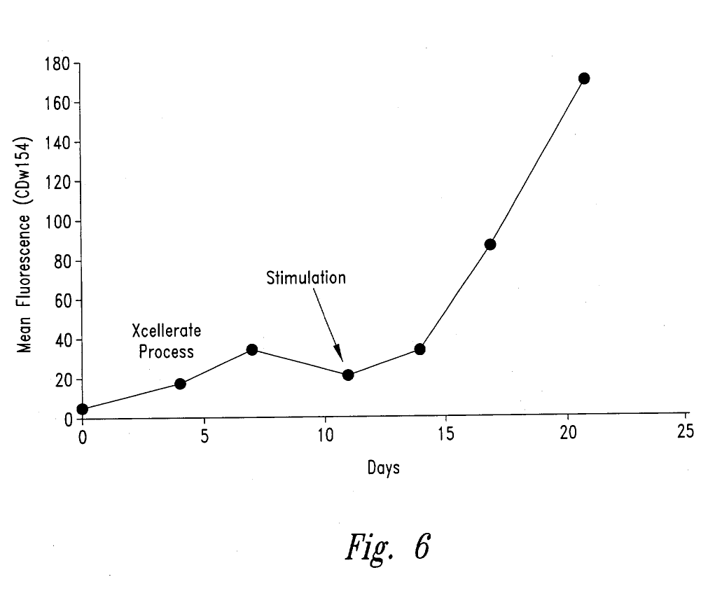

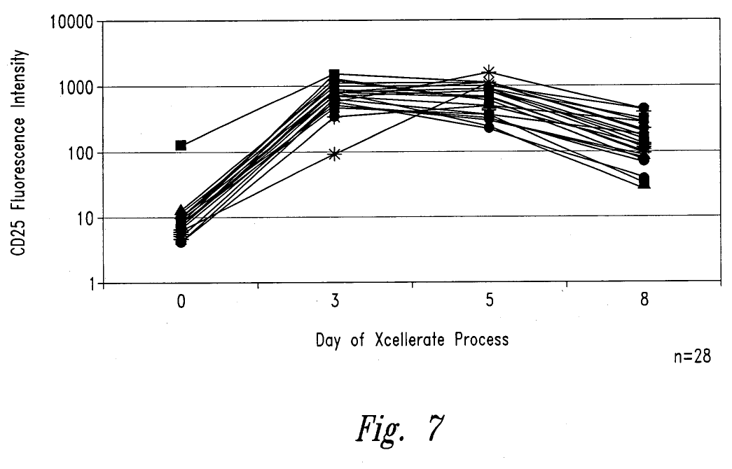

Expression levels of CDw137 (41BB), CD154 (CD40L), and CD25 on Xcellerated T cells were analyzed by flow cytometry, and the mean fluorescence plotted, as shown in FIG. 5, FIG. 6, and FIG. 7, respectively. As shown in FIG. 5, Expression levels of CDw137 (4-1 BB) increase and peak at day 4 and then decrease gradually. Following re-stimulations, expression of CDw137 increased rapidly. FIG. 6 demonstrates that expression of CD40L increases gradually until about day 7 and then decreases. Following re-stimulation, however, levels of CD40L increase rapidly and to much higher levels than during the initial stimulation. Levels of CD25 increased until about day 3 and then decreased gradually until day 8 (the last time point analyzed) (FIG. 7).

### Example 2

### Comparative Morphology of Mature Dendritic Cells Generated by Standard Approaches Versus XCELLERATE Process

Methods: Fresh leukapheresis product was washed twice in RPMI with 5% human serum. PBMCs were resuspended and cultured in RPMI supplemented with 5% human AB serum, L-glutamine, pen-strep at 1×, and cultured in tissue culture flasks at 1×10⁶ cells/cm² at 37° C. and 5% CO₂ for about 1-2 hours or until the monocytes adhered to the flask. Following this period, the non-adherent cells were gently removed and the media replaced with fresh RPMI as above with the addition of 100 ng/mL GM-CSF and 60 ng/ml IL-4. Thereafter, the media was replaced every 2 days. On day 6 of culture, 3 separate culture conditions were set up: In one flask, day 2 or day 3 (day 2/3) XCELLERATE™ activated T cells generated from the same leukapheresis product (see patent application Ser. No 09/794,230) were added to the immature DC at a ratio of 5 T cells/DC in media containing GM-CSF and IL-4. One flask contained immature DC in media containing only GM-CSF and IL-4 and CD40L 1 ng/mL was added to the media containing GM-CSF and IL-4 in a third flask. After 24 and 48 hours, cells were examined under a light microscope. Immature DC cultured in the presence of day 2/3 XCELLERATE™ T cells have dendritic processes that are significantly more prominent, an important morphological indication of maturity. Immature DCs cultured using standard methods of GM-CSF+IL-4 or GM-CSF+IL-4+CD40L demonstrate dendritic processes that are less prominent. Thus, the XCELLERATE™ activated T cells lead to a significantly improved maturation of the immature DCs over the current standard methods of maturing DCs.

### Example 3

### Cytokine-Free Generation of Immature Dendritic Cells

Immature DC were generated from monocytes (isolated as described in Example 2) by incubation in the presence of Xcellerated T cell supernatants as described in detail below.

Cell Preparation

Cryopreserved PBMC were washed 3 times with PBS and adjusted to 5% human serum and incubated one hour at room temperature. Cells were filtered through a 80 μM net filter, counted and set up for positive-selection in a 1000 ml Lifecell bag. Cells (1,000×10⁶ cells) were suspended in 50 ml PBS and adjusted to 5% human serum. 10 ml washed Xcellerate beads (Dynabeads CD3/CD28 T Cell Expander) (washed using the same medium) were added to the cells and the bag was rotated edgewise for 30 min. 150 ml PBS (5% human serum) was added. The bag was put on a Maxsep device and unbound cells were drained off at 30 ml/min (setting of 12) into the 300 ml bag. 1000 ml of BASIC MEDIUM (XCELLERATE; see below)+100 IU/ML IL-2 was added to the culture bag and bead-bound cells were drained into the 3000 ml lifecell culture bag. Cells were incubated at 37° C. in the incubator for 3 days to generate stimulated T cell supernatant.

The Unselected (non-bound) flow through cells were counted and used for the monocyte culture setup, described briefly below.

Supernatant from T cell cultures were collected at 72 hours and stored frozen for later use.

Basic Medium

X-Vivo 15 supplemented with 5% human serum, 2 mM L-glut (2 mM), 20 mM HEPES

Part 1: Culture Supernatant Collection

Day 3 Culture supernatant of T cells positively selected and stimulated with Xcellerate 3×28 beadswas collected (experiment NDa-171—Bag A). Cells and beads were removed by centrifugation at 3000 rpm for 10 minutes. Supernatant was frozen at −85° C.

Part 2: Monocyte Set Up

Cells from the flow-through fraction (unbound) were concentrated by centrifugation and resuspended in Culture Media. Monocyte concentration (%) was estimated to be approximately 20%, based upon flow cytometric analysis. 10×10⁶ total cells were plated in each T-25 flask in 4.0 ml and incubated at 37° C. in a CO₂ incubator.

Timepoint #1. After 1 hour, flasks were washed to remove non-adherent cells and 7.0 ml of either Xcellerate activated T cell supernatant or culture medium alone was added (see below).

Timepoint #2. Repeat above the next day.

Part 3: Staining and Flow Cytometry

After 48-72 hours of culture in the presence of activated-T cell supernatants, monocyte culture cells were stained as indicated below for phenotypic analysis by flow cytometry.

| | FITC* | PE** | Tricolor |
|---|---|---|---|
| 1 | anti-CD3 | anti-CD19 + anti-CD20 | Anti-CD14 |
| 3 | anti-lineage | anti-CD83 | anti-HLA-DR |
| 4 | anti-lineage | anti-CD86 | anti-HLA-DR |

*FITC: Fluoresceine isothiocyanate
**PE: Phyco-Erythrin

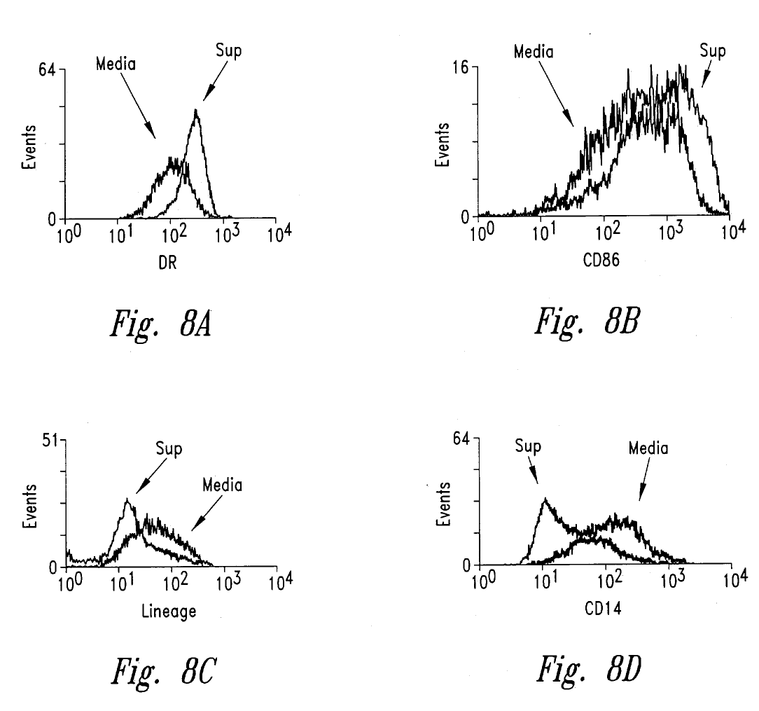

As shown in FIG. 8, HLA-DR and CD86 expression increased and Lineage and CD14 expression decreased in the presence of activated T cell supernatants, expression profiles indicative of the generation of immature DC.

### Example 4

### Induction of DC Maturation by Standard Approaches Versus XCELLERATE T Cells

Methods: Leukapheresis product was washed three times in PBS. Cells were cultured immediately in RPMI 1640 supplemented with 1% human AB serum in tissue culture flasks at 0.5-1×10⁶ cells/cm² at 37° C. under 5% CO₂ for 1-2 hours. The nonadherent cells were removed by gentle rinsing of the tissue culture flasks with the medium. The adherent cells were cultured further in the presence of 100 ng/ml GM-CSF and 60 ng/ml IL-4 in the medium for 6 days. On days 2 and 4 of culture, one half of culture medium was replaced with fresh medium containing 200 ng/ml GM-CSF and 120 ng/ml of IL-4. On day 6 loosely adherent immature DC were harvested. Cells were resuspended in fresh medium containing 100 ng/ml GM-CSF, 60 ng/ml IL-4 and 100 U/ml IL-2 and cultured at 0.5-1×10⁵/cm² in the presence of day 3 Xcellerate T cells at a T cell:immature DC ratio of 4:1. As a positive or negative control for the DC maturation process, cells were cultured in the presence or absence of 100 ng/ml CD40L plus 1,000 U/ml IFN-γ without T cells. After 24 hours cells were harvested and their phenotype was examined by FACS analysis.

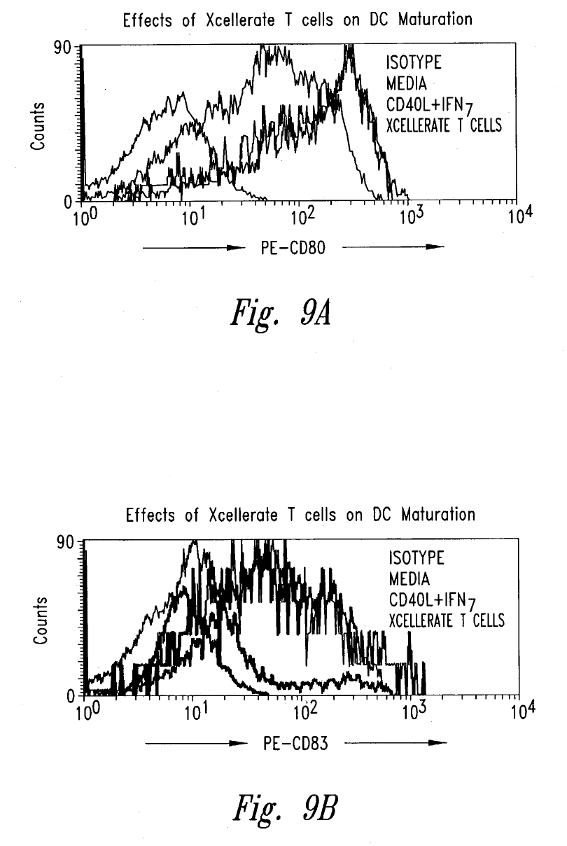

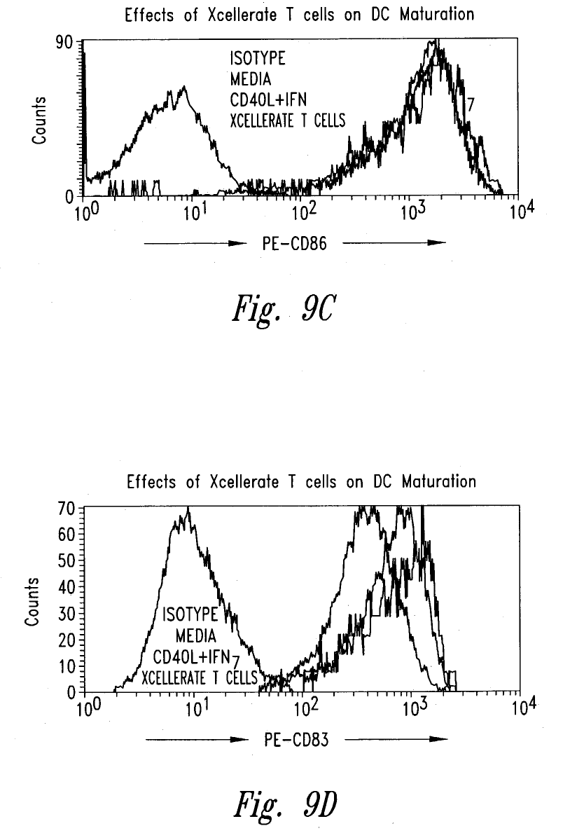

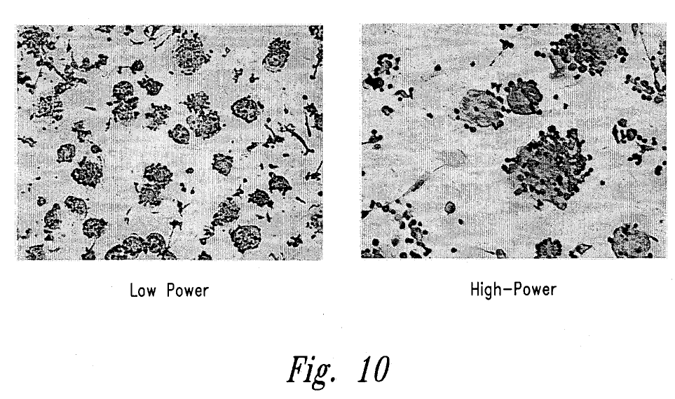

Results: FIG. 9 shows that the upregulation of cell surface CD80, CD83, CD86 and HLA-DR was markedly higher on DCs resulting from co-culture with Xcellerate T cells than that of DC resulting from culture in the absence of T cells (negative control). The upregulation of the markers by co-culturing with Xcellerate activated T cells was comparable with that obtained with the combination of CD40L and IFN-γ (positive control). DC generated by co-culturing with Xcellerated T cels also expressed DC-LAMP. Allostimulatory activity of mature DC generated by co-culturing with Xcellerated T Cells is comparable with that of mature DC generated in the presence of CD40L/IFN-γ. The results indicate that co-culturing of immature DC with day 3 Xcellerate T cells induces maturation of DC. Anti-CD154 antibody partially blocks the maturation of DC induced by co-culturing with Xcellerated T Cells, suggesting a role for CD40L expressed on the surface of activated T cells in guiding DC maturation.

### Example 5

Generation of Multinucleated Cells By Co-Culture of XCELLERATE T Cells with Monocytes

Methods: Fresh leukapheresis product was washed twice in X-Vivo 15 supplemented with 5% human serum. PBMCs were resuspended and cultured in X-Vivo supplemented with 1% human AB serum, 2 mM L-glutamine, 50 U/ml penicilin and 50 μg/ml streptomycin, and cultured in tissue culture flasks at 1×10⁶ cells/cm² at 37° C. and 5% CO₂ for about 1-2 hours or until the monocytes adhered to the flask. Following this period, the non-adherent cells were gently removed and the medium replaced with fresh medium as above. On day 1 or 2 of culture, day 2-3 Xcellerate T cells were added to the culture at a T cell:monocyte ratio of at least 1:1. Cells were then cultured for an additional 3-4 days. As shown in FIG. 10, multinucleated cells appeared in the culture. Without being bound by theory, the composition of these multinucleated cells may be either maturing monocytic cells that have engulfed apoptosed/apoptosing Xcellerated T cells. Alternatively, these cells may be osteoclasts.

### Example 6

### Generation of Dendritic Cells by Culture of Monocytes with Day 3 XCELLERATE™ T Cell Supernatant (TCS)

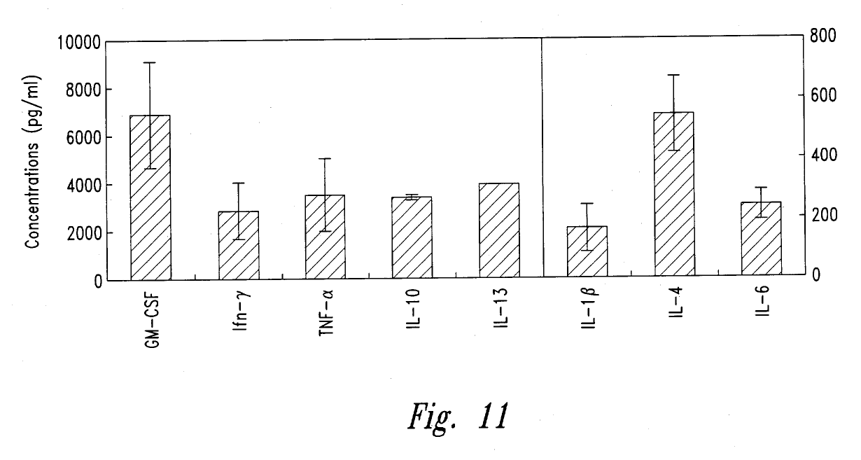

As described in Example 1, XCELLERATE™ T cells produce IL-4, IFN-γ, IL-2, TNF-α, and express CDw137 and CD154. XCELLERATE™ T cells also produce GM-CSF, IL-10, IL-13, IL-1 and IL-6 (FIG. 11), and moderate levels of TGF-β. As described below, when cultured in day 3 XCELLERATE™ T cell supernatant (TCS) or XCELLERATE™ cells, monocytes mature into DC.

Methods: Fresh leukapheresis product was washed twice in X-Vivo 15 supplemented with 5% human serum. PBMC were then aliquoted and frozen.

On day −3, an aliquot was thawed, and the Xcellerate activation of T cells was initiated as described in Example 1. On day −2, another aliquot was thawed and the PBMCs were resuspended and cultured in X-Vivo supplemented with 1% human AB serum, 2 mM L-glutamine, 50 U/ml penicilin and 50 μg/ml streptomycin, and cultured in tissue culture flasks at 1×10⁶ cells/cm² at 37° C. and 5% CO₂ for about 1-2 hours or until the monocytes adhered to the flask. Following this period, the non-adherent cells were gently removed and the media replaced with fresh media as above. On day 0, supernatant was collected from the day 3 Xcellerate T cells and aliquoted. One aliquot was added to the day 2 adherent monocyte cultures, and the remaining aliquotes were frozen. On days 1-8, an aliquot was thawed and used to replace the medium of the adherent monocyte cultures. On each of these days, an aliquot of cells was removed and analyzed by flow cytometry and microscopy. Monocyte control cultures were also set up in parallel with medium alone, medium supplemented with GM-CSF and IL-4 and medium supplemented with GM-CSF and IL-4, with IFN-γ and CD40L added on day 5.

The experiments showed that Xcellerate TCS harvested on Day 3 of T cell activation, induced the differentiation of monocytes into immature DC with the phenotype of Lin-/CD80-/CD83-/CD86+/DR+. Allostimulatory activity of immature DC generated in the presence of Xcellerate TCS was less than that of immature DC generated with GM-CSF/IL-4, but significantly higher than that of monocytes cultured with medium alone.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method for maturing dendritic cells, comprising:
(a) providing a population of cells wherein at least a portion thereof comprises immature dendritic cells; and
(b) exposing the population of cells to activated T cells or supernatant therefrom, thereby inducing maturation.

2. The method of claim 1 wherein the immature dendritic cells are generated from a source of precursor cells selected from the group consisting of leukapheresis product, peripheral blood, lymph node, skin, GALT, tonsil, thymus, tissue biopsy, tumor, spleen, skin, bone marrow, cord blood, CD34⁺ cells, monocytes, and adherent cells.

3. The method of claim 2 wherein the immature dendritic cells are generated by exposing the precursor cells to activated T cells or supernatant therefrom.

4. The method of claim 1 wherein the immature dendritic cells comprise dendritic/tumor cell fusions.

5. The method of claim 4 wherein the tumor cells used to generate the dendritic/tumor cell fusions are from a cancer.

6. The method of claim 5 wherein the cancer is selected from the group consisting of

7. The method of claim 1 wherein the immature dendritic cells are generated from a source of precursor cells selected from the group consisting of leukapheresis product, peripheral blood, lymph node, skin, GALT, tonsil, thymus, tissue biopsy, tumor, spleen, bone marrow, cord blood, CD34⁺ cells, monocytes, and adherent cells by exposing said precursor cells to one or more cytokines.

8. The method of claim 7 wherein the cytokines comprise GM-CSF.

9. The method of claim 7 wherein the cytokines comprise IL-4.

10. The method of claim 7 wherein the cytokines comprise GM-CSF and IL-4.

11. The method of claim 7 wherein the cytokines comprise IL-13.

12. The method of claim 7 wherein the cytokines comprise GM-CSF and IL-13.

13. The method of claim 7 wherein the source of precursor cells comprises leukapheresis product.

14. The method of claim 7 wherein the source of precursor cells comprises peripheral blood.

15. The method of claim 7 wherein the source of precursor cells comprises bone marrow.

16. The method of claim 7 wherein the source of precursor cells comprises cord blood.

17. The method of claim 7 wherein the source of precursor cells comprises CD34⁺ cells.

18. The method of claim 7 wherein the source of precursor cells comprises monocytes.

19. The method of claim 7 wherein the source of precursor cells comprises adherent cells.

20. The method of claim 1 wherein the immature dendritic cells are generated from a source of precursor cells selected from the group consisting of leukapheresis product, peripheral blood, lymph node, skin, GALT, tonsil, thymus, tissue biopsy, tumor, spleen, skin, bone marrow, cord blood, CD34⁺ cells, monocytes, and adherent cells by exposing said precursor cells to one or more cytokines and activated T cells or supernatant therefrom.

21. The method according to claim 1 wherein the immature dendritic cells are loaded with antigen through gene modification or by exposing the immature dendritic cells to a source of antigen selected from the group consisting of protein, peptides, tumor lysate, and apoptotic bodies.

22. The method according to claim 21 wherein the source of antigen comprises protein.

23. The method according to claim 21 wherein the source of antigen comprises peptides and/or polypeptides

24. The method according to claim 21 wherein the source of antigen comprises tumor lysates.

25. The method according to claim 21 wherein the source of antigen comprises apoptotic bodies.

26. The method according to claim 21 wherein the source of antigen comprises irradiated tumor cells from a tumor or a cell line.

27. The method according to claim 1 wherein the dendritic cells are genetically modified.

28. The method of claim 1 wherein the activated T cells comprise a T cell line.

29. The method of claim 1 wherein the activated T cells are generated by cell surface moiety ligation comprising:
(a) providing a population of cells wherein at least a portion thereof comprises T cells; and
(b) exposing the population of cells to an agent that induces activation of said T cells.

30. The method of claim 29 wherein the agent comprises anti-T cell receptor antibodies.

31. The method of claim 29 wherein the agent comprises anti-CD3 antibodies.

32. The method of claim 29 wherein the agent comprises anti-CD28 antibodies.

33. The method of claim 29 wherein the agent comprises anti-CD3 and anti-CD28 antibodies.

34. The method of claim 1 wherein the activated T cells are generated by simultaneous T cell concentration and cell surface moiety ligation, comprising:
(a) providing a population of cells wherein at least a portion thereof comprises T cells;
(b) exposing the population of cells to a surface, wherein the surface has attached thereto one or more agents that ligate a cell surface moiety of at least a portion of the T cells and stimulates at least a portion of T cells.
(c) applying a force that predominantly drives T cell concentration and T cell surface moiety ligation, thereby inducing T cell stimulation.

35. A population of mature dendritic cells generated according to the method of claim 1.

36. A population of mature dendritic cells according to claim 35 wherein the dendritic cells are fused to tumor cells to form dendritic/tumor cell fusions.

37. A composition comprising the dendritic/tumor cell fusions according to claim 36 and a pharmaceutically acceptable excipient.

38. A method for stimulating an immune response in a mammal comprising, administering to the mammal the composition of claim 37.

39. A method for reducing the presence of cancer cells in a mammal comprising, exposing the cells to the composition of claim 37.

40. A method for inhibiting the development of a cancer in a mammal, comprising administering to the mammal the composition of claim 37.

41. A composition comprising the dendritic cells according to claim 35 and a pharmaceutically acceptable excipient.

42. A composition according to claim 41 wherein the dendritic cells are genetically modified.

43. A method for stimulating an immune response in a mammal comprising, administering to the mammal the composition of claim 41.

44. The method of claim 43 wherein the immune response comprises the activation of T cells in the mammal.

45. A method for ameliorating an immune response dysfunction in a mammal comprising administering to the mammal the composition of claim 41.

46. A method for reducing the presence of cancer cells in a mammal comprising, exposing the cells to the composition of claim 41.

47. The method of claim 46 wherein the cancer cells are from a cancer selected from the group consisting of melanoma, non-Hodgkin's lymphoma, Hodgkin's disease, leukemia, acute lymphoblastic leukemia, acute myelogenous leukemia, chronic myelogenous leukemia, and chronic lymphocytic leukemia.

48. The method of claim 46 wherein the cancer comprises leukemia.

49. A method for reducing the presence of an infectious organism in a mammal comprising, administering to the mammal the composition of claim 41.

50. A method for inhibiting the development of a cancer in a mammal, comprising administering to the mammal the composition of claim 41.

51. The method of claim 50 wherein the cancer is selected from the group consisting of melanoma, non-Hodgkin's lymphoma, Hodgkin's disease, leukemia, acute lymphoblastic leukemia, acute myelogenous leukemia, chronic myelogenous leukemia, and chronic lymphocytic leukemia.

52. The method of claim 50 wherein the cancer comprises leukemia.

53. A method for inhibiting the development of an infectious disease in a mammal, comprising administering to the mammal the composition of claim 41.

54. A composition comprising dendritic cells and activated T cells wherein the dendritic cells have been matured by exposure to activated T cells or supernatant therefrom ex vivo.

55. The composition of claim 54, further comprising a pharmaceutically acceptable excipient.

56. A method for stimulating an immune response in a mammal, comprising administering to the mammal the composition of claim 55.

57. A method for inhibiting the development of a cancer in a mammal, comprising administering to the mammal the composition of claim 55.

58. The method of claim 57 wherein the cancer is selected from the group consisting of melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, pancreatic cancer, esophageal cancer, brain cancer, lung cancer, ovarian cancer, cervical cancer, multiple myeloma, hepatomocellular carcinoma, acute lymphoblastic leukemia, acute myelogenous leukemia, chronic myelogenous leukemia, and chronic lymphocytic leukemia.

59. The method of claim 57 wherein the cancer comprises leukemia.

60. A method for inhibiting the development of an infectious disease in a mammal, comprising administering to the mammal the composition of claim 55.

61. A method for reducing the presence of cancer cells in a mammal, comprising administering to the mammal a composition comprising, dendritic cells matured by activated T cells or supernatant therefrom ex vivo, activated T cells, and a pharmaceutically acceptable excipient.

62. The method of claim 61 wherein the cancer cells are selected from the group consisting of a melanoma, non-Hodgkin's lymphoma, Hodgkin's disease, leukemia, plasmocytoma, sarcoma, glioma, thymoma, breast cancer, prostate cancer, colo-rectal cancer, kidney cancer, renal cell carcinoma, pancreatic cancer, esophageal cancer, brain cancer, lung cancer, ovarian cancer, cervical cancer, multiple myeloma, hepatocellular carcinoma, acute lymphoblastic leukemia, acute myelogenous leukemia, chronic myelogenous leukemia, and chronic lymphocytic leukemia.

63. The method of claim 61 wherein the cancer cells comprise leukemia.

64. A method for generating mature dendritic cells in vivo comprising, administering to a mammal a composition comprising activated T cells.

65. A method for generating mature dendritic cells, comprising:
(a) generating immature dendritic cells in vitro from a source of precursor cells by a method selected from the group consisting of:
i. exposing the precursor cells to GM-CSF and IL-4;
ii. exposing the precursor cells to GM-CSF and IL-13;
iii. exposing the precursor cells to activated T cells or supernatant therefrom;
iv. exposing the precursor cells to GM-CSF and IL-4 and activated T cells or supernatant therefrom; and
v. exposing the precursor cells to GM-CSF and IL-13 and activated T cells or supernatant therefrom;
(b) administering to a mammal the immature dendritic cells of part (a), and;
(c) administering to the mammal activated T cells, thereby inducing in vivo maturation of the immature dendritic cells.

66. The method of claim 65 wherein the source of precursor cells is selected from the group consisting of leukapheresis product, peripheral blood, lymph node, skin, GALT, tonsil, thymus, tissue biopsy, tumor, spleen, skin, bone marrow, cord blood, CD34⁺ selected cells, monocytes, and adherent cells.

67. The method of claim 65 wherein the source of precursor cells is leukapheresis product.

68. The method of claim 65 wherein the source of precursor cells is peripheral blood.

69. The method of claim 65 wherein the source of precursor cells is bone marrow.

70. The method of claim 65 wherein the source of precursor cells is cord blood.

71. The method of claim 65 wherein the source of precursor cells is CD34⁺ cells.

72. The method of claim 65 wherein the source of precursor cells is monocytes.

73. The method of claim 65 wherein the source of precursor cells is adherent cells.

74. A method for generating mature dendritic cells, comprising:
(a) obtaining a population of cells from a mammal wherein at least a portion thereof comprises precursor dendritic cells;
(b) exposing said portion of cells in vitro to GM-CSF and IL-4 or IL-13 to generate immature dendritic cells; and
(c) exposing said immature dendritic cells in vitro to a population of activated T cells or supernatant therefrom for a sufficient period of time to achieve desired maturation.

75. The method of claim 65 wherein the precursor cells are isolated from peripheral blood.

76. The method of claim 65 wherein the precursor cells are isolated from leukapheresis product.

77. The method of claim 76 wherein the activated T cells are generated by a method comprising, exposing the population of T cells to an anti-CD3 antibody and a ligand which binds an accessory molecule on the surface of the T cells, under conditions appropriate for activation of the T cells.

78. The method of claim 76 wherein said activated T cells are generated by a method comprising:
(a) exposing the population of T cells to an anti-CD3 antibody which is immobilized on a solid phase surface; and;
(b) stimulating an accessory molecule on the surface of the T cells with an anti-CD28 antibody, wherein said anti-CD28 antibody is immobilized on the same solid phase surface as the anti-CD3 antibody, thereby inducing activation and proliferation of the T cells.

79. The method of claim 78 wherein the activated T cells generated comprise T cells that have proliferated.

80. The method of claim 78 wherein the activated T cells generated comprise T cells that secrete cytokines.

81. A method for expanding dendritic/tumor cell fusions comprising exposing the dendritic/tumor cell fusions to activated T cells or supernatant therefrom.
